(19) United States
(12) Patent Application Publication
(10) Pub. No.: US 2009/0099214 A1
(43) Pub. Date: Apr. 16, 2009

(54) Organic Compounds

(75) Inventors: Robin Alec Fairhurst, West Sussex (GB); Roger John Taylor, West Sussex (GB)

Correspondence Address: NOVARTIS INSTITUTES FOR BIOMEDICAL RESEARCH, INC., 400 TECHNOLOGY SQUARE, CAMBRIDGE, MA, 02139, US

(73) Assignee: Novartis AG

(21) Appl. No.: 12/297,404

(22) PCT Filed: Apr. 19, 2007
(86) PCT No.: PCT/EP2007/003434

§ 371 Date: Oct. 16, 2008

(30) Foreign Application Priority Data
Apr. 21, 2006 (GB) 0607948.7

(51) Int. Cl. A61K 31/52; C07D 473/00; A61P 29/00

(52) U.S. Cl. 514/263.22; 544/277

(57) ABSTRACT

A compound of formula (I) or stereoisomers or pharmaceutically acceptable salts thereof, and their preparation and use as pharmaceuticals

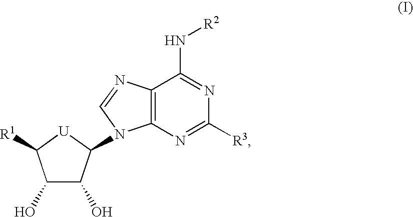

wherein U, R¹, R² and R³ are as defined herein.

This invention relates to organic compounds, their preparation and use as pharmaceuticals.

An aspect of the present invention provides compounds of formula (I) or stereoisomers or pharmaceutically acceptable salts thereof,

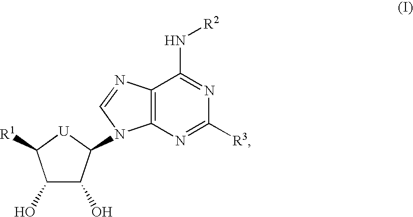

wherein
U is selected from CH₂ and O, with the proviso that when U is O then R¹ is not a N-bonded substituent;
R¹ is a 3- to 12-membered heterocyclic group containing from 1 to 4 ring nitrogen atoms and optionally containing from 1 to 4 other heteroatoms selected from the group consisting of oxygen and sulfur, that group being optionally substituted by oxo, C₁-C₈-alkoxy, C₆-C₁₀-aryl, R^{1a} or by C₁-C₈-alkyl optionally substituted by hydroxyl, or
R¹ is —NH₂, —NH—C₁-C₈-alkylcarbonyl, —NH—C₃-C₈-cycloalkylcarbonyl, —NHSO₂—C₁-C₈-alkyl, —NH—C₇-C₁₄-aralkylcarbonyl or —NHC(═O)—C(═O)—NH—C₁-C₈-alkyl optionally substituted by R^{1a}, or
R¹ is selected from CH₂OH, CH₂—O—C₁-C₈-alkyl, C(O)—O—C₁-C₈-alkyl, C(O)NH₂, and C(O)—NH—C₁-C₈-alkyl;
R^{1a} is a 3- to 12-membered heterocyclic ring containing at least one ring heteroatom selected from the group consisting of nitrogen, oxygen and sulfur, said 3- to 12-membered heterocyclic ring being optionally substituted by halo, cyano, oxo, hydroxy, carboxy, amino, nitro, C₁-C₈-alkyl, C₁-C₈-alkylsulfonyl, aminocarbonyl, C₁-C₈-alkylcarbonyl or C₁-C₈-alkoxy optionally substituted by aminocarbonyl;
R² is C₁-C₈-alkyl substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, a C₃-C₁₅-carbocyclic group optionally substituted by O—C₇-C₁₄ aralkyl, C₃-C₁₅-carbocyclic group, O—C₁-C₈-alkyl, C₂-C₈-alkenyl, C₂-C₈-alkynyl or C₁-C₈-alkyl, O—C₁-C₈-alkyl, —SO₂—C₁-C₈-alkyl, a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl, with the proviso that R² is not 2,2,diphenyl-ethyl, or
R² is a C₃-C₁₅-carbocyclic group optionally substituted by O—C₇-C₁₄ aralkyl, C₃-C₁₅-carbocyclic group, O—C₁-C₈-alkyl, or C₁-C₈-alkyl, or
R² is a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl;
R³ is hydrogen, halo, C₂-C₈-alkenyl, C₂-C₈-alkynyl, C₁-C₈-alkoxycarbonyl, C₁-C₈-alkyl optionally substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, or
R³ is amino optionally substituted by C₃-C₈-cycloalkyl optionally substituted by amino, hydroxy, C₇-C₁₄-aralkyloxy, —SO₂—C₆-C₁₀-aryl or —NH—C(═O)—NH—R^{3c}, or
R³ is amino substituted by R^{3a}, —R^{3a}—C₇-C₁₄-aralkyl or a C₅-C₁₅-carbocyclic group optionally substituted by OH, C₁-C₈-alkyl or C₁-C₈-alkoxycarbonyl, or
R³ is aminocarbonyl optionally substituted by R^{3b}, or
R³ is C₁-C₈-alkylamino optionally substituted by OH, R^{3b}, amino, di(C₁-C₈-alkyl)amino, —NH—C(═O)—C₁-C₈-alkyl, —NH—SO₂—C₁-C₈-alkyl, —NH—C(═O)—NH—R^{3c}, —NH—C(═O)—NH—C₁-C₈-alkyl-R^{3b}, a C₅-C₁₅-carbocyclic group or by C₆-C₁₀-aryl optionally substituted by C₆-C₁₀-aryloxy, or
R³ is C₁-C₈-alkylaminocarbonyl or C₃-C₈-cycloalkylamino-carbonyl optionally substituted by amino, C₁-C₈-alkylamino, di(C₁-C₈-alkyl)amino or —NH—C(═O)—NH—R^{3d}, or
R³ is a 3- to 12-membered heterocyclic group containing from 1 to 4 ring nitrogen atoms and optionally containing from 1 to 4 other heteroatoms selected from the group consisting of oxygen and sulfur, that group being optionally substituted by 0-3R⁴;
R^{3a} and R^{3b} are each independently a 3- to 12-membered heterocyclic group containing at least one ring heteroatom selected from the group consisting of nitrogen, oxygen and sulfur; optionally substituted by halo, cyano, oxo, OH, carboxy, nitro, C₁-C₈-alkyl, C₁-C₈-alkylcarbonyl, OH—C₁-C₈-alkyl, C₁-C₈-haloalkyl, amino-C₁-C₈-alkyl, amino(OH)C₁-C₈-alkyl and C₁-C₈-alkoxy optionally substituted by aminocarbonyl;
R^{3c} is a 5- or 6-membered heterocyclic group containing at least one ring heteroatom selected from the group consisting of nitrogen, oxygen and sulfur, which is optionally substituted by a 5- or 6-membered heterocyclic group containing at least one ring heteroatom selected from the group consisting of nitrogen, oxygen and sulfur;
R^{3d} are independently a 5- or 6-membered heterocyclic ring containing at least one ring heteroatom selected from the group consisting of nitrogen, oxygen and sulfur, said 5- or 6-membered heterocyclic ring being optionally substituted by halo, cyano, oxo, OH, carboxy, amino, nitro, C₁-C₈-alkyl, C₁-C₈-alkylsulfonyl, aminocarbonyl, C₁-C₈-alkylcarbonyl, C₁-C₈-alkoxy optionally substituted by aminocarbonyl, or a 5- or 6-membered heterocyclic ring containing at least one ring heteroatom selected from the group consisting of nitrogen, oxygen and sulfur, said ring also being optionally substituted by halo, cyano, oxo, OH, carboxy, amino, nitro, C₁-C₈-alkyl, C₁-C₈-alkylsulfonyl, aminocarbonyl, C₁-C₈-alkylcarbonyl, C₁-C₈-alkoxy optionally substituted by aminocarbonyl;
R⁴ is selected from OH, C₁-C₈-alkyl optionally substituted by OH, C₁-C₈-alkoxy, C₇-C₁₄-aralkyl optionally substituted with OH, O—C₁-C₈-alkyl, halogen C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, O—C₁-C₈-alkyl or -halogen, NR^{4a}R^{4b}, NHC(O)R^{4c}, NHS(O)₂R^{4d}, NHS(O)₂R^{4e}, NR^{4f}C(O)NR^{4e}R^{4h}, NR⁴ⁱC(O)OR^{4j}, C₁-C₈-alkylcarbonyl, C₁-C₈-alkoxycarbonyl, di(C₁-C₈-alkyl)aminocarbonyl, COOR^{4k}, C(O)R^{4l}, and a 3- to 12-membered heterocyclic group containing at least one ring heteroatom selected from the group consisting of nitrogen, oxygen and sulfur optionally substituted by COOR^{4p};
R^{4a}, R^{4b}, R^{4c}, R^{4f}, R^{4h} and R⁴ⁱ are, independently, H, or C₁-C₈-alkyl;
R^{4d}, R^{4e}, and R^{4j} are, independently, C₁-C₈-alkyl or a 3- to 12-membered heterocyclic group containing at least one ring heteroatom selected from the group consisting of nitrogen, oxygen and sulfur, optionally substituted by 0-3R⁵;
R^{4k} is H, C₁-C₈-alkyl, C₆-C₁₀-aryl or a 3- to 12-membered heterocyclic group containing at least one ring heteroatom selected from the group consisting of nitrogen, oxygen and sulfur;
R^{4l} is C₁-C₈-alkyl, C₆-C₁₀-aryl, NHR⁶ or a 3- to 12-membered heterocyclic group containing at least one ring heteroatom selected from the group consisting of nitrogen, oxygen and sulfur;
R⁵ is selected from OH, C₁-C₈-alkyl optionally substituted by OH, SO₂R¹⁰, C₇-C₁₄-aralkyl optionally substituted with OH, O—C₁-C₈-alkyl, C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, NR^{5a}R^{5b}, NHC(O)R^{5c}, NHS(O)₂R^{5d}, NHS(O)₂R^{5e}, NR^{5f}C(O)NR^{5g}R^{5h}, NR⁵ⁱC(O)OR^{5j}, C₁-C₈-alkylcarbonyl, C₁-C₈-alkoxycarbonyl, di(C₁-C₈-alkyl)aminocarbonyl, COOR^{5k}, C(O)R^{5l}, C(O)NHR^{5m} or a 3- to 12-membered heterocyclic group containing at least one ring heteroatom selected from the group consisting of nitrogen, oxygen and sulfur, optionally substituted by 0-3R⁷;
R^{5a}, R^{5b}, R^{5c}, R^{5f}, R^{5h} and R⁵ⁱ are, independently, H, C₁-C₈-alkyl or C₆-C₁₀-aryl;
R^{5d}, R^{5e}, R^{5g}, R^{5j} and R^{5m} are, independently, C₁-C₈-alkyl or a 3- to 12-membered heterocyclic group containing at least one ring heteroatom selected from the group consisting of nitrogen, oxygen and sulfur, optionally substituted by COOR⁸;
R^{5k} is H, C₁-C₈-alkyl, C₆-C₁₀-aryl or a 3- to 12-membered heterocyclic group containing at least one ring heteroatom selected from the group consisting of nitrogen, oxygen and sulfur,
R^{5l} is C₁-C₈-alkyl, C₆-C₁₀-aryl or a 3- to 12-membered heterocyclic group containing at least one ring heteroatom selected from the group consisting of nitrogen, oxygen and sulfur, optionally substituted by COOR⁹;
R⁶ is COOR^{6a} or a 3- to 12-membered heterocyclic group containing at least one ring heteroatom selected from the group consisting of nitrogen, oxygen and sulfur, optionally substituted by COOR^{6b};
R^{6a}, R^{6b}, R⁷, R⁸ and R⁹ are selected from H, C₁-C₈-alkyl and C₇-C₁₄-aralkyl; and
R¹⁰ is C₁-C₈-alkyl optionally substituted by halogen, C₆-C₁₀-aryl optionally substituted by OH, C₁-C₈-alkyl, O—C₁-C₈-alkyl or -halogen.

According to formula (I), R¹ is suitably —NH—C₁-C₈-alkylcarbonyl, —NH—C₃-C₈-cycloalkylcarbonyl, —NHSO₂—C₁-C₈-alkyl, —NH—C₇-C₁₄-aralkylcarbonyl or —NHC(═O)—C(═O)—NH—C₁-C₈-alkyl. Preferably R¹ is —NH—C₁-C₈-alkylcarbonyl such as —NH—C(O)—ethyl.

According to formula (I), R1 is also suitably a 5- or 6-membered heterocyclic group such as tetrazole. The tetrazole is suitably substituted by C₁-C₈-alkyl (e.g. methyl or ethyl). According to formula (I), R² is suitably C₁-C₈-alkyl substituted by OH, a C₃-C₁₅-carbocyclic group such as fluorene or one or two C₆-C₁₀ aryls such as phenyl that can be optionally substituted by OH, O—C₁-C₈-alkyl, CN, halogen, SO₂NH₂, SCH₃, a C₆-C₁₀-aryl, or O—C₇-C₁₄-aralkyl

According to formula (I), R² is also suitably an alkyl substituted by C₃-C₁₅-carbocyclic group such as a cyclopropyl group which is substituted by C₂-C₈-alkenyl.

According to formula (I), R² is also suitably a C₃-C₁₅-carbocyclic group optionally substituted by a C₃-C₁₅-carbocyclic group such as a cyclopentyl group, a C₇-C₁₄ aralkyl such as benzyl, or O—C₇-C₁₄-aralkyl.

According to formula (I), R² is also suitably a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl. Preferably the 3- to 12-membered heterocyclic group is a 5-to-6-membered heterocyclic group such as pyrrolidine or piperidine. This heterocyclic group can be optionally substituted by C₇-C₁₄ aralkyl such as a benzyl group or optionally substituted by 3- to 12-membered heterocyclic group containing from 1 to 4 ring nitrogen atoms and optionally containing from 1 to 4 other heteroatoms selected from the group consisting of oxygen and sulfur, such as a pyridine group.

According to formula (I), R³ is suitably hydrogen, halo, C₂-C₈-alkenyl, C₂-C₈-alkynyl, C₁-C₈-alkoxycarbonyl, C₁-C₈-alkyl optionally substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl. Preferably R³ is halo such as chlorine. R³ is also suitably C₁-C₈ alkyl substituted by OH and a phenyl ring.

According to formula (I), R³ is also suitably a 3- to 12-membered heterocyclic group containing from 1 to 4 ring nitrogen atoms and optionally containing from 1 to 4 other heteroatoms selected from the group consisting of oxygen and sulfur, that group being optionally substituted by 0-3R⁴. Preferably the 3- to 12-membered heterocyclic group is pyrrolidine. The pyrrolidine is suitably substituted by —NH₂, or —NHC(O)NH-3- to 12-membered heterocyclic group such as pyrrolidine, piperidine or pyridine. When the 3- to 12-membered group of —NHC(O)NH-3- to 12-membered heterocyclic group is piperidine, the piperidine can be further substituted by a 3- to 12-membered heterocyclic group such as pyridine.

According to formula (I), R³ is also suitably C₁-C₈-alkylamino substituted by 3- to 12-membered heterocyclic group (e.g. piperidine, pyrrolidine, and pyrazole). The 3- to 12-membered heterocyclic group can be substituted by a C₁-C₈-alkyl group, such as a methyl or ethyl group.

According to formula (I), R³ is also suitably an amino substituted by a C₃-C₁₅ carbocyclic group (e.g. cyclohexyl) which can be substituted by an amine.

Another aspect of the invention provides compounds of formula (I) or stereoisomers or pharmaceutically acceptable salts thereof,

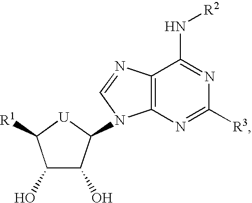

wherein
U is selected from CH₂ and O, with the proviso that when U is O then R¹ is not a N-bonded substituent
R¹ is —NH₂, —NH—C₁-C₈-alkylcarbonyl, —NH—C₃-C₈-cycloalkylcarbonyl, —NHSO₂—C₁-C₈-alkyl, —NH—C₇-C₁₄-aralkylcarbonyl or —NHC(═O)—C(═O)—NH—C₁-C₈-alkyl optionally substituted by R^{1a}, or
R¹ is a 3- to 12-membered heterocyclic group containing from 1 to 4 ring nitrogen atoms and optionally containing from 1 to 4 other heteroatoms selected from the group consisting of oxygen and sulfur, that group being optionally substituted by C₁-C₈-alkyl optionally substituted by hydroxyl;
R^{1a} is a 5- or 6-membered heterocyclic ring containing at least one ring heteroatom selected from the group consisting of nitrogen, oxygen and sulphur, said 5- or 6-membered heterocyclic ring being optionally substituted by halo, cyano, oxo, OH, carboxy, amino, nitro, C₁-C₈-alkyl, C₁-C₈-alkylsulfonyl, aminocarbonyl, C₁-C₈-alkylcarbonyl or C₁-C₈-alkoxy optionally substituted by aminocarbonyl;
R² is C₁-C₈-alkyl substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, a C₃-C₁₅-carbocyclic group optionally substituted by O—C₇-C₁₄ aralkyl, C₃-C₁₅-carbocyclic group, O—C₁-C₈-alkyl, C₂-C₈-alkenyl, C₂-C₈-alkynyl or C₁-C₈-alkyl, O—C₁-C₈-alkyl, —SO₂—C₁-C₈-alkyl, a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl, or
R² is a C₃-C₁₅-carbocyclic group optionally substituted by O—C₇-C₁₄ aralkyl, C₃-C₁₅-carbocyclic group, O—C₁-C₈-alkyl, or C₁-C₈-alkyl, or
R² is a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl;
R³ is hydrogen, halo, C₂-C₈-alkenyl, C₂-C₈-alkynyl, C₁-C₈-alkoxycarbonyl, C₁-C₈-alkyl optionally substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, or
R³ is amino optionally substituted by C₃-C₁₅-carbocyclic group optionally substituted by amino, or
R³ is C₁-C₈-alkylamino optionally substituted by OH, R^{3b}, amino, di(C₁-C₈-alkyl)amino, —NH—C(═O)—C₁-C₈-alkyl, —NH—SO₂—C₁-C₈-alkyl, —NH—C(═O)—NH—R^{3c}, —NH—C(═O)—NH—C₁-C₈-alkyl-R^{3b}, a C₅-C₁₅-carbocyclic group or by C₆-C₁₀-aryl optionally substituted by C₆-C₁₀-aryloxy, or
R³ is a 3- to 12-membered heterocyclic group containing from 1 to 4 ring nitrogen atoms and optionally containing from 1 to 4 other heteroatoms selected from the group consisting of oxygen and sulfur, that group being optionally substituted by 0-3R⁴;
R^{3b} are each independently a 3- to 12-membered heterocyclic group containing at least one ring heteroatom selected from the group consisting of nitrogen, oxygen and sulfur; optionally substituted by halo, cyano, oxo, OH, carboxy, nitro, C₁-C₈-alkyl, C₁-C₈-alkylcarbonyl, OH—C₁-C₈-alkyl, C₁-C₈-haloalkyl, amino-C₁-C₈-alkyl, amino(OH)C₁-C₈-alkyl and C₁-C₈-alkoxy optionally substituted by aminocarbonyl;
R^{3C} is a 5- or 6-membered heterocyclic group containing at least one ring heteroatom selected from the group consisting of nitrogen, oxygen and sulfur, which is optionally substituted by a 5- or 6-membered heterocyclic group containing at least one ring heteroatom selected from the group consisting of nitrogen, oxygen and sulfur;
R⁴ is selected from OH, C₁-C₈-alkyl optionally substituted by OH, C₁-C₈-alkoxy, C₇-C₁₄-aralkyl optionally substituted with OH, O—C₁-C₈-alkyl, halogen C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, O—C₁-C₈-alkyl or -halogen, NR^{4a}R^{4b}, NHC(O)R^{4c}, or NR^{4f}C(O)NR^{4e}R^{4h};
R^{4a}, R^{4b}, R^{4c}, R^{4f}, and R^{4h} are, independently, H, or C₁-C₈-alkyl;
R^{4e} is C₁-C₈-alkyl or a 3- to 12-membered heterocyclic group containing at least one ring heteroatom selected from the group consisting of nitrogen, oxygen and sulfur, optionally substituted by 0-3R⁵;
R⁵ is selected from OH, C₁-C₈-alkyl optionally substituted by OH, SO₂R¹⁰, C₇-C₁₄-aralkyl optionally substituted with OH, O—C₁-C₈-alkyl, C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, NR^{5a}R^{5b}, NHC(O)R^{5c}, NHS(O)₂R^{5d}, NHS(O)₂R^{5e}, NR^{5f}C(O)NR^{5g}R^{5h}, NR⁵ⁱC(O)OR^{5j}, C₁-C₈-alkylcarbonyl, C₁-C₈-alkoxycarbonyl, di(C₁-C₈-alkyl)aminocarbonyl, COOR^{5k}, C(O)R^{5l}, C(O)NHR^{5m} or a 3- to 12-membered heterocyclic group containing at least one ring heteroatom selected from the group consisting of nitrogen, oxygen and sulfur, optionally substituted by 0-3R⁷;
R^{5a}, R^{5b}, R^{5c}, R^{5f}, R^{5h} and R⁵ⁱ are, independently, H, C₁-C₈-alkyl or C₆-C₁₀-aryl;
R^{5d}, R^{5e}, R^{5g}, R^{5j} and R^{5m} are, independently, C₁-C₈-alkyl or a 3- to 12-membered heterocyclic group containing at least one ring heteroatom selected from the group consisting of nitrogen, oxygen and sulfur, optionally substituted by COOR⁸;
R^{5k} is H, C₁-C₈-alkyl, C₆-C₁₀-aryl or a 3- to 12-membered heterocyclic group containing at least one ring heteroatom selected from the group consisting of nitrogen, oxygen and sulfur;
R^{5l} is C₁-C₈-alkyl, C₆-C₁₀-aryl or a 3- to 12-membered heterocyclic group containing at least one ring heteroatom selected from the group consisting of nitrogen, oxygen and sulfur, optionally substituted by COOR⁹;
R⁶ is COOR^{6a} or a 3- to 12-membered heterocyclic group containing at least one ring heteroatom selected from the group consisting of nitrogen, oxygen and sulfur, optionally substituted by COOR^{6b};
R^{6a}, R^{6b}, R⁷, R⁸ and R⁹ are selected from H, C₁-C₈-alkyl and C₇-C₁₄-aralkyl; and
R¹⁰ is C₁-C₈-alkyl optionally substituted by halogen, C₆-C₁₀-aryl optionally substituted by OH, C₁-C₈-alkyl, O—C₁-C₈-alkyl or -halogen.

Another aspect of the invention provides compounds of formula (I) or stereoisomers or pharmaceutically acceptable salts thereof,
R¹ is —NH—C₁-C₈-alkylcarbonyl, —NH—C₃-C₈-cycloalkylcarbonyl, —NHSO₂—C₁-C₈-alkyl, —NH—C₇-C₁₄-aralkylcarbonyl or —NHC(═O)—C(═O)—NH—C₁-C₈-alkyl, or
R¹ is a 3- to 12-membered heterocyclic group containing from 1 to 4 ring nitrogen atoms and optionally containing from 1 to 4 other heteroatoms selected from the group consisting of oxygen and sulfur, that group being optionally substituted by C₁-C₈-alkyl optionally substituted by hydroxyl;
R² is C₁-C₈-alkyl substituted by OH, a C₃-C₁₅-carbocyclic group, C₆-C₁₀ aryl optionally substituted by OH, O—C₁-C₈-alkyl, CN, halogen, SO₂NH₂, SCH₃, a C₆-C₁₀-aryl, or O—C₇-C₁₄-aralkyl, or
R² is C₁-C₈-alkyl substituted by C₃-C₁₅-carbocyclic group optionally substituted by C₂-C₈-alkenyl, or
R² is a C₃-C₁₅-carbocyclic group optionally substituted by a C₃-C₁₅-carbocyclic group, a C₇-C₁₄ aralkyl, or O—C₇-C₁₄-aralkyl, or
R² is a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl; and
R³ is hydrogen, halo, C₂-C₈-alkenyl, C₂-C₈-alkynyl, C₁-C₈-alkoxycarbonyl, C₁-C₈-alkyl optionally substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, or
R³ is a 3- to 12-membered heterocyclic group containing from 1 to 4 ring nitrogen atoms and optionally containing from 1 to 4 other heteroatoms selected from the group consisting of oxygen and sulfur, that group being optionally substituted by 0-3R⁴, or
R³ is C₁-C₈-alkylamino substituted by 3- to 12-membered heterocyclic group optionally substituted by a C₁-C₈-alkyl group, or
R³ is an amino substituted by a C₃-C₁₅-carbocyclic group optionally substituted by an amine.

### DEFINITIONS

Terms used in the specification have the following meanings:

“Optionally substituted” means the group referred to can be substituted at one or more positions by any one or any combination of the radicals listed thereafter.

“Halo” or “halogen”, as used herein, may be fluorine, chlorine, bromine or iodine. Preferably halo is chlorine.

“Hydroxy”, as used herein, is OH.

“C₁-C₈-alkyl”, as used herein, denotes straight chain or branched alkyl having 1-8 carbon atoms. Preferably C₁-C₈-alkyl is C₁-C₄-alkyl.

“C₁-C₈-alkoxy”, or as used herein, denotes straight chain or branched alkoxy having 1-8 carbon atoms. Preferably, C₁-C₈-alkoxy is C₁-C₄-alkoxy.

“C₃-C₈-cycloalkyl”, as used herein, denotes cycloalkyl having 3-8 ring carbon atoms, e.g., a monocyclic group, such as a cyclopropyl, cyclobutyl, cyclopentyl, cyclohexyl, cycloheptyl or cyclooctyl, any of which can be substituted by one or more, usually one or two, C₁-C₄-alkyl groups; or a bicyclic group, such as bicycloheptyl or bicyclooctyl.

“C₁-C₈-alkylamino” and “di(C₁-C₈-alkyl)amino”, as used herein, denote amino substituted respectively by one or two C₁-C₈-alkyl groups as hereinbefore defined, which may be the same or different.

“C₁-C₈-alkylcarbonyl” and “C₁-C₈-alkoxycarbonyl”, as used herein, denote C₁-C₈-alkyl or C₁-C₈-alkoxy, respectively, as hereinbefore defined attached by a carbon atom to a carbonyl group.

“C₆-C₁₀-aryl”, as used herein, denotes a monovalent carbocyclic aromatic group that contains 6-10 carbon atoms and which may be, e.g., a monocyclic group, such as phenyl; or a bicyclic group, such as naphthyl.

“C₇-C₁₄-aralkyl”, as used herein, denotes alkyl, e.g., C₁-C₄-alkyl, as hereinbefore defined, substituted by C₆-C₁₀-aryl as hereinbefore defined. Preferably, C₇-C₁₄-aralkyl is C₇-C₁₀-aralkyl, such as phenyl-C₁-C₄-alkyl.

“C₁-C₈-alkylaminocarbonyl” and “C₃-C₈-cycloalkylaminocarbonyl” as used herein denote C₁-C₈-alkylamino and C₃-C₈-cycloalkylamino respectively as hereinbefore defined attached by a carbon atom to a carbonyl group. Preferably C₁-C₈-alkylaminocarbonyl and C₃-C₈-cycloalkyl-aminocarbonyl are C₁-C₄-alkylaminocarbonyl and C₃-C₈-cycloalkylaminocarbonyl respectively.

“C₃-C₁₅-carbocyclic group” as used herein denotes a carbocyclic group having 3 to 15 ring carbon atoms, for example a monocyclic group, either aromatic or non-aromatic, such as a cyclopentyl, cyclohexyl, cycloheptyl, cyclooctyl or phenyl, or a bicyclic group such as bicyclooctyl, bicyclononyl, bicyclodecyl, indanyl or indenyl, again any of which can be substituted by one or more, usually one or two, C₁-C₄-alkyl groups. Preferably the C₅-C₁₅-carbocyclic group is a C₅-C₁₀-carbocyclic group, especially phenyl, cyclohexyl or indanyl. The C₅-C₁₅-carbocyclic group can unsubstituted or substituted. Preferred substituents on the heterocyclic ring include halo, cyano, OH, carboxy, amino, aminocarbonyl, nitro, C₁-C₁₀-alkyl, C₁-C₁₀-alkoxy and C₃-C₁₀-cycloalkyl, especially OH or amino.

“3- to 12-membered heterocyclic ring containing at least one ring heteroatom selected from the group consisting of nitrogen, oxygen and sulfur”, as used herein, may be, e.g., furan, pyrrole, pyrrolidine, pyrazole, imidazole, triazole, isotriazole, tetrazole, thiadiazole, isothiazole, oxadiazole, pyridine, piperidine, pyrazine, oxazole, isoxazole, pyrazine, pyridazine, pyrimidine, piperazine, pyrrolidine, morpholino, triazine, oxazine or thiazole. Preferred heterocyclic rings include piperazine, pyrrolidine, morpholino, imidazole, isotriazole, pyrazole, tetrazole, thiazole, triazole, thiadiazole, pyridine, piperidine, pyrazine, furan, oxazole, isoxazole, oxadiazole and azetidine. The 3- to- 12-membered heterocyclic ring can be unsubstituted or substituted.

“5- or 6-membered heterocyclic group containing at least one ring heteroatom selected from the group consisting of nitrogen, oxygen and sulfur” as used herein may be, for example, a saturated or unsaturated heterocyclic group such as furanyl, pyrrolyl, pyrrolidinyl, pyrazolyl, imidazolyl, triazolyl, isotriazolyl, tetrazolyl, thiadiazolyl, isothiazolyl, oxadiazolyl, pyridinyl, piperidinyl, pyrazinyl, oxazolyl, isoxazolyl, pyrazinyl, pyridazinyl, pyrimidinyl, piperazinyl, pyrrolidinyl, morpholinyl, triazinyl, oxazinyl or thiazolyl. Preferred 5- or 6-membered heterocyclic groups include pyrazolyl, imidazolyl, pyrrolidinyl, pyridinyl and piperidinyl. The 5- or 6-membered heterocyclic group can be unsubstituted or substituted. Preferred substituents include halo, cyano, oxo, OH, carboxy, amino, nitro, C₁-C₈-alkyl (optionally substituted by hydroxy), C₁-C₈-alkylsulfonyl, aminocarbonyl, C₁-C₈-alkylcarbonyl, C₁-C₈-alkoxycarbonyl, and C₁-C₈-alkoxy optionally substituted by aminocarbonyl. Especially preferred substituents include chloro, cyano, carboxy, amino, C₁-C₈-alkoxycarbonyl, C₁-C₄-alkoxy and C₁-C₄-alkyl optionally substituted by OH.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

Especially preferred specific compounds of formula (I) are those described hereinafter in the Examples.

### Salts and Isomers

The compounds represented by formula (I) are capable of forming acid addition salts, particularly pharmaceutically acceptable acid addition salts. Pharmaceutically acceptable acid addition salts of the compound of formula I include those of inorganic acids, for example, hydrohalic acids such as hydrofluoric acid, hydrochloric acid, hydrobromic acid or hydroiodic acid, nitric acid, sulfuric acid, phosphoric acid; and organic acids, for example aliphatic monocarboxylic acids such as formic acid, acetic acid, trifluoroacetic acid, propionic acid and butyric acid, aliphatic hydroxy acids such as lactic acid, citric acid, tartaric acid or malic acid, dicarboxylic acids such as maleic acid or succinic acid, aromatic carboxylic acids such as benzoic acid, p-chlorobenzoic acid, diphenylacetic acid, para-biphenyl benzoic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, cinnamic acids such as 3-(2-naphthalenyl)propenoic acid, para-methoxy cinnamic acid or para-methyl cinnamic acid, and sulfonic acids such as methanesulfonic acid or benzenesulfonic acid. These salts may be prepared from compounds of formula I by known salt-forming procedures.

Compounds of formula (I) which contain acidic, e.g. carboxyl, groups, are also capable of forming salts with bases, in particular pharmaceutically acceptable bases such as those well known in the art; suitable such salts include metal salts, particularly alkali metal or alkaline earth metal salts such as sodium, potassium, magnesium or calcium salts, or salts with ammonia or pharmaceutically acceptable organic amines or heterocyclic bases such as ethanolamines, benzylamines or pyridine. These salts may be prepared from compounds of formula I by known salt-forming procedures.

Stereoisomers are those compounds where there is an asymmetric carbon atom. The compounds exist in individual optically active isomeric forms or as mixtures thereof, e.g., as diastereomeric mixtures. The present invention embraces both individual optically active R and S isomers, as well as mixtures thereof. Individual isomers can be separated by methods well known to those skilled in the art, e.g. chiral high performance liquid chromatography (HPLC).

Tautomers are one of two or more structural isomers that exist in equilibrium and are readily converted from one isomeric form to another.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

### Synthesis

Another embodiment of the present invention, provides a process for the preparation of compounds of formula (I), in free or pharmaceutically acceptable salt form, which comprises the steps of:

(i) reacting a compound of formula (II)

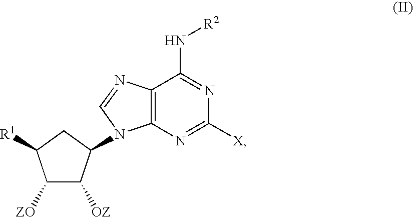

wherein
R¹, and R² are as defined in Claim 1;
Z is H or a protecting group; and
X is a leaving group,
   with a compound of formula (III)

H—R³ (III),

wherein
R³ is as defined in Claim 1; and
removing any protecting groups and recovering the resultant compound of formula (I), in free or pharmaceutically acceptable salt form.

The compound of formula (III) may be prepared by reacting a compound of formula (IV)

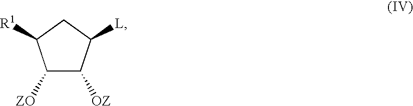

wherein
R¹ and Z are as defined in Claim 1; and
L represents a leaving group or a protected derivative thereof with a 2,6-dihalopurine, e.g., 2,6-dichloropurine,
   to provide a compound of formula (V)

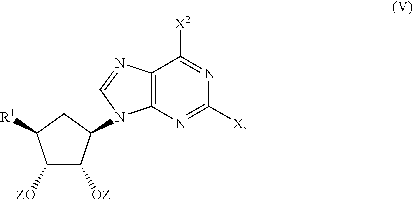

wherein
R¹ and Z are defined in Claim 1; and
X and X² are halogen.

Compound of formula (V) can be reacted with R²NH₂ under conventional conditions to provide compound of formula (II).

The compounds of formula (I) can be prepared, e.g., using the reactions and techniques described below and in the Examples. The compounds of formula (I) can be prepared by the processes described in patent application PCT/EP2005/011344. The reactions may be performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of formula (I) into another compound of formula (I). Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such as alkylation, acylation, halogenation or oxidation. Such manipulations are well-known in the art, and many reference works summarize procedures and methods for such manipulations. Some reference works which gives examples and references to the primary literature of organic synthesis for many functional group manipulations, as well as other transformations commonly used in the art of organic synthesis are March's Organic Chemistry, 5^{th} Edition, Wiley and Chichester, Eds. (2001); Comprehensive Organic Transformations, Larock, Ed., VCH (1989); Comprehensive Organic Functional Group Transformations, Katritzky et al. (series editors), Pergamon (1995); and Comprehensive Organic Synthesis, Trost and Fleming (series editors), Pergamon (1991). It will also be recognized that another major consideration in the planning of any synthetic route in this field is the judicious choice of the protecting group used for protection of the reactive functional groups present in the compounds described in this invention. Multiple protecting groups within the same molecule can be chosen such that each of these protecting groups can either be removed without removal of other protecting groups in the same molecule, or several protecting groups can be removed using the same reaction step, depending upon the outcome desired. An authoritative account describing many alternatives to the trained practitioner is Protective Groups In Organic Synthesis, Greene and Wuts, Eds., Wiley and Sons (1999). It is understood by those skilled in the art that only combinations of substituents that are chemically possible are embodiments of the present invention.

### Pharmaceutical Use

Compounds of formula I and their pharmaceutically acceptable salts are useful as pharmaceuticals. In particular, they activate the adenosine A2a receptor activation, i.e. they act as A2a receptor agonists. Their properties as A2a agonists may be demonstrated using the method described by L in the following test procedures described in J. J P. Murphree Hannon et al in Molecular (1998) Dr Pharmacology 61, 455-462 (2002)ug Development Research 43, 214-224.

Compounds of the Examples herein below have Kᵢ values below 1.0 μM in the above method. For example, the compounds of Examples 7, 8, 17, 19, and 38 have Kᵢ values of 0.003, 0.002, 0.011, 0.008, and 0.007 μM respectively.

Having regard to their activation of the adenosine A2a receptor, compounds of formula (I) in free or pharmaceutically acceptable salt form, hereinafter alternately referred to as “agents of the invention”, are useful in the treatment of conditions which are mediated by response to the activation of the adenosine A2a receptor, particularly inflammatory or allergic conditions. Treatment in accordance with the invention may be symptomatic or prophylactic.

Accordingly, agents of the invention are useful in the treatment of inflammatory or obstructive airways diseases, resulting, for example, in reduction of tissue damage, airways inflammation, bronchial hyperreactivity, remodelling or disease progression. Inflammatory or obstructive airways diseases and conditions to which the present invention is applicable include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include bronchiectasis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Other inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. cortico-steroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant from any previously administered symptomatic asthma therapy.

Having regard to their anti-inflammatory activity, in particular in relation to inhibition of eosinophil activation, agents of the invention are also useful in the treatment of eosinophil related disorders, e.g. eosinophilia, in particular eosinophil related disorders of the airways (e.g. involving morbid eosinophilic infiltration of pulmonary tissues) including hyper-eosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Agents of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin.

Agents of the invention may also be used for the treatment of other diseases or conditions, in particular diseases or conditions having an inflammatory component, for example, treatment of diseases and conditions of the eye such as conjunctivitis, keratoconjunctivitis sicca, and vernal conjunctivitis, diseases affecting the nose including allergic rhinitis, and inflammatory disease in which autoimmune reactions are implicated or having an autoimmune component or aetiology, including autoimmune haematological disorders (e.g. haemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary billiary cirrhosis, uveitis (anterior and posterior), keratoconjunct-ivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy).

Further, agents of the invention may also be used for the treatment of cystic fibrosis, pulmonary hypertension, pulmonary fibrosis, inflammatory bowel syndrome, wound healing, diabetic nephropathy as described in WO 05/107463, reduction of inflammation in transplanted tissue as described in US 2005/182018, inflammatory diseases caused by pathogenic organisms as described in WO 03/086408, and cardiovascular conditions as described in WO 03/029264.

Also, the agents of the invention may be used to assess the severity of coronary artery stenosis as described in WO 00/078774 and useful in conjunction with radioactive imaging agents to image coronary activity and useful in adjunctive therapy with angioplasty as described in WO 00/78779.

Agents of the invention are also useful in combination with a protease inhibitor for prevention of organ ischaemia and reperfusion injury as described in WO 05/003150, and in combination with an integrin antagonist for treating platelet aggregation as described in WO 03/090733.

Agents of the invention are also useful in promoting wound healing in bronchial epithelial cells as described in AJP-Lung 290: 849-855.

Other diseases or conditions which may be treated with agents of the invention include diabetes, e.g. diabetes mellitus type I (juvenile diabetes) and diabetes mellitus type II, diarrheal diseases, ischemia/reperfusion injuries, retinopathy, such as diabetic retinopathy or hyperbaric oxygen-induced retinopathy, conditions characterised by elevated intraocular pressure or secretion of ocular aqueous humor, such as glaucoma, ischemic tissue/organ damage from reperfusion, bedsores, as agents for promoting sleep, as agents for treating demyelinating diseases, eg multiple sclerosis and as neuroprotective agents for eg, cerebral haemorrhagic injury and spinal cord ischaemi-reperfusion injury.

The effectiveness of an agent of the invention in inhibiting inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; Cernadas et al (999) Am. J. Respir. Cell Mol. Biol. 20:1-8; and Fozard et al (2002) European Journal of Pharmacological 438, 183-188.

The agents of the invention are also useful as co-therapeutic agents for use in combination with other drug substances such as anti-inflammatory, bronchodilatory, antihistamine or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance.

Accordingly the invention includes a combination of an agent of the invention as hereinbefore described with an anti-inflammatory, bronchodilatory, antihistamine or anti-tussive drug substance, said agent of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTB4 antagonists such as BIIL 284, CP-195543, DPC11870, LTB4 ethanolamide, LY 293111, LY 255283, CGS025019C, CP-195543, ONO-4057, SB 209247, SC-53228 and those described in U.S. Pat. No. 5,451,700; LTD4 antagonists such include montelukast, pranlukast, zafirlukast, accolate, SR2640, Wy-48,252, ICI 198615, MK-571, LY-171883, Ro 24-5913 and L-648051; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID(™) CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; adenosine A_{2B} receptor antagonists such as those described in WO 02/42298; and beta-2 adrenoceptor agonists such as albuterol (salbutamol), metaproterenol, terbutaline, salmeterol fenoterol, procaterol, and especially, formoterol, carmoterol and pharmaceutically acceptable salts thereof, and compounds (in free or salt or solvate form) of formula I of WO 0075114, which document is incorporated herein by reference, preferably compounds of the Examples thereof, especially a compound of formula

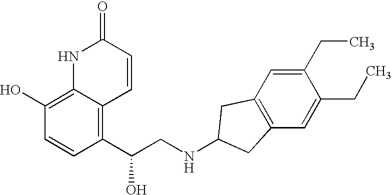

and pharmaceutically acceptable salts thereof, as well as compounds (in free or salt or solvate form) of formula I of WO 04/16601, and also compounds of EP 1440966, JP 05025045, WO 93/18007, WO 99/64035, US 2002/0055651, US 2005/0133417, US 2005/5159448, WO 01/42193, WO 01/83462, WO 02/66422, WO 02/70490, WO 02/76933, WO 03/24439, WO 03/42160, WO 03/42164, WO 03/72539, WO 03/91204, WO 03/93219, WO 03/99764, WO 04/16578, WO 04/22547, WO 04/32921, WO 04/33412, WO 04/37768, WO 04/37773, WO 04/37807, WO 04/39762, WO 04/39766, WO 04/45618 WO 04/46083, WO 04/80964, EP1460064, WO 04/087142, WO 04/089892, EP 01477167, US 2004/0242622, US 2004/0229904, WO 04/108675, WO 04/108676, WO 05/033121, WO 05/040103, WO 05/044787, WO 05/058867, WO 05/065650, WO 05/066140, WO 05/07908, US 2005/5159448, US 2005/171147, WO 05/077361, WO 05/084640, WO 05/089760, WO 05/090287, WO 05/090288, WO 05/092860, WO 05/092887, US 2005/182091, US 2005/209227, US 2005/215542, US 2005/215590, EP 1574501, US 05/256115, WO 05/102350 and US 05/277632.

Suitable bronchodilatory drugs include anticholinergic or antimuscarinic agents, in particular ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, US 2005/171147, US 2005/182091, WO 01/04118, WO 02/00652, WO 02/51841, WO 02/53564, WO 03/00840, WO 03/33495, WO 03/53966, WO 03/87094, WO 04/018422, WO 04/05285 and WO 05/077361.

Suitable dual anti-inflammatory and bronchodilatory drugs include dual beta-2 adrenoceptor agonist/muscarinic antagonists such as those disclosed in US 2004/0167167, US 2004/0242622, US 2005/182092, WO 04/74246 WO 04/74812, WO 04/089892 and US 05/256114.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in JP 2004107299, WO 03/099807 and WO 04/026841.

Other useful combinations of agents of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-amin-ium chloride (TAK-770), and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

In accordance with the foregoing, the invention also provides a method for the treatment of a condition mediated byresponsive to activation of the adenosine A2a receptor, for example an inflammatory or allergic condition, particularly an inflammatory or obstructive airways disease, which comprises administering to a subject, particularly a human subject, in need thereof a compound of formula I in free form or in the form of a pharmaceutically acceptable salt. In another aspect the invention provides a compound of formula II, in free form or in the form of a pharmaceutically acceptable salt, for use in the manufacture of a medicament for the treatment of a condition mediated byresponsive to activation of the adenosine A2a receptor, particularly an inflammatory or obstructive airways disease.

### Formulation and Administration

The agents of the invention may be administered by any appropriate route, e.g. orally, for example in the form of a tablet or capsule; parenterally, for example intravenously; by inhalation, for example in the treatment of inflammatory or obstructive airways disease; intranasally, for example in the treatment of allergic rhinitis; topically to the skin, for example in the treatment of atopic dermatitis; or rectally, for example in the treatment of inflammatory bowel disease.

In a further aspect, the invention also provides a pharmaceutical composition comprising a compound of formula (I) in free form or in the form of a pharmaceutically acceptable salt, optionally together with a pharmaceutically acceptable diluent or carrier therefor. The composition may contain a co-therapeutic agent such as an anti-inflammatory, broncho-dilatory, antihistamine or anti-tussive drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro-fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula II having a particle diameter up to 10 microns, optionally together with a diluent or carrier, such as lactose, of the desired particle size distribution and a compound that helps to protect against product performance deterioration due to moisture e.g. magnesium stearate. When the composition comprises a nebulised formulation, it preferably contains, for example, the compound of formula (I) either dissolved, or suspended, in a vehicle containing water, a co-solvent such as ethanol or propylene glycol and a stabiliser, which may be a surfactant.

The invention includes (A) a compound of formula (I) in inhalable form, e.g. in an aerosol or other atomisable composition or in inhalable particulate, e.g. micronised, form, (B) an inhalable medicament comprising a compound of formula (I) in inhalable form; (C) a pharmaceutical product comprising a compound of formula (I) in inhalable form in association with an inhalation device; and (D) an inhalation device containing a compound of formula (I) in inhalable form.

Dosages of compounds of formula (I) employed in practising the present invention will of course vary depending, for example, on the particular condition to be treated, the effect desired and the mode of administration. In general, suitable daily dosages for administration by inhalation are of the order of 0.005 to 10 mg, while for oral administration suitable daily doses are of the order of 0.05 to 100 mg.

The invention is illustrated by the following Examples.

Preferred Compounds of Formula I

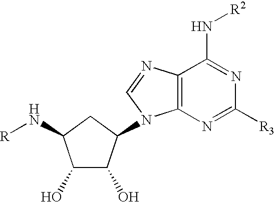

are shown in Table 1 below.

TABLE 1

| Ex. | R | R² | R³ | MH+ |
|---|---|---|---|---|
| 1 | 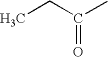 | 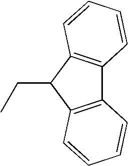 | —Cl | 519.3 |
| 2 | 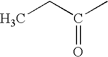 | 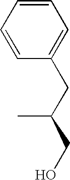 | —Cl | 475.3 |
| 3 | 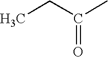 | 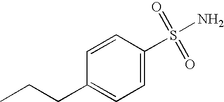 | —Cl | 524.2 |
| 4 | 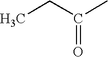 | 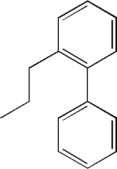 | —Cl | 521.3 |
| 5 | 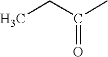 | 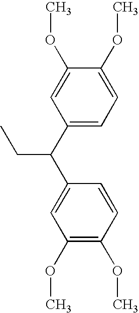 | —Cl | 641.4 |
| 6 | 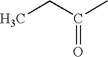 | 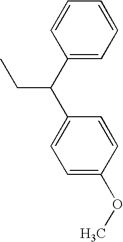 | —Cl | 551.3 |
| 7 | 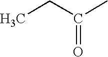 | 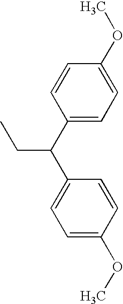 | 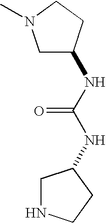 | 743.5 |
| 8 | 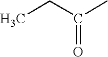 | 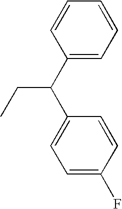 | 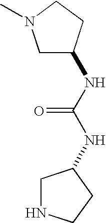 | 701.5 |
| 9 | 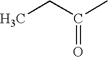 | 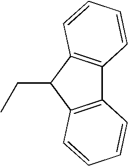 | 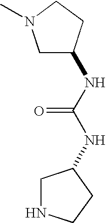 | 681.5 |
| 10 | 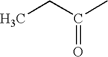 | 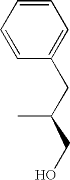 | 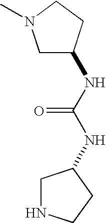 | 637.4 |
| 11 | 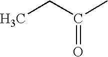 | 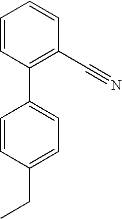 | 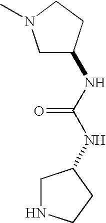 | 694.5 |
| 12 | 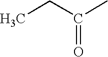 | 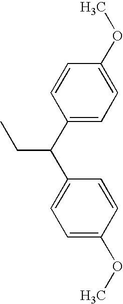 | 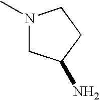 | 631.4 |
| 13 | 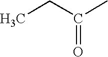 | 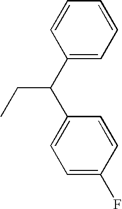 | 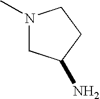 | 589.4 |
| 14 | 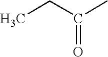 | 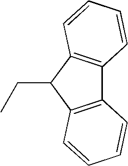 | 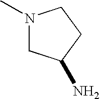 | 569.4 |
| 15 | 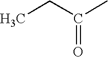 | 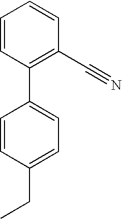 | 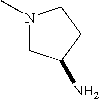 | 528.3 |
| 16 | 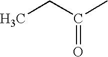 | 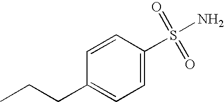 | 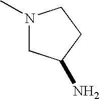 | 574.3 |
| 17 | 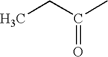 | 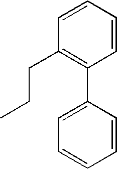 | 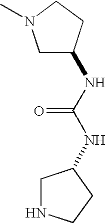 | 683.5 |
| 18 | 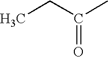 | 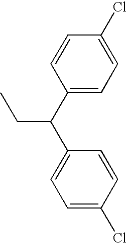 | 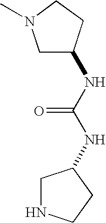 | 751.4 |
| 19 | 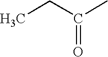 | 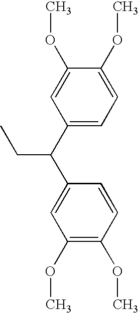 | 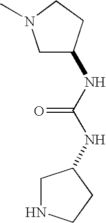 | 803.5 |
| 20 | 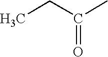 | 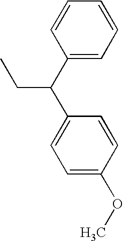 | 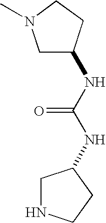 | 713.5 |
| 21 | 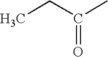 | 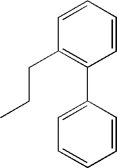 | 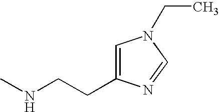 | 624.4 |
| 22 | 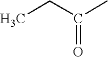 | 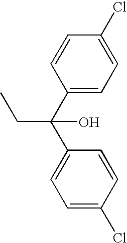 | 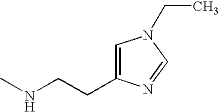 | 708.3 |
| 23 | 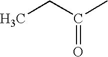 | 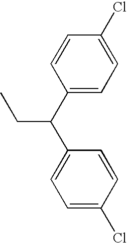 | 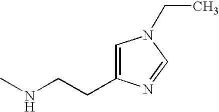 | 629.3 |
| 24 | 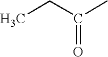 | 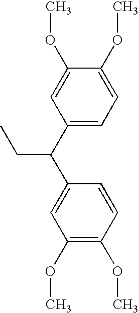 | 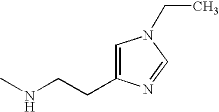 | 744.5 |
| 25 | 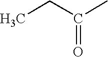 | 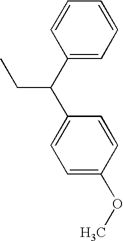 | 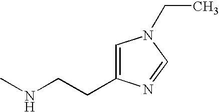 | 654.4 |
| 26 | 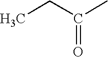 | 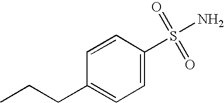 | 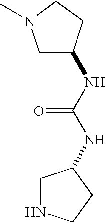 | 686.3 |
| 27 | 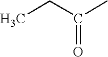 | 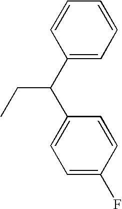 | —Cl | 539.3 |
| 28 | 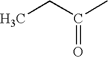 | 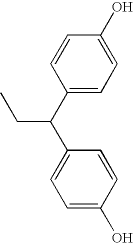 | 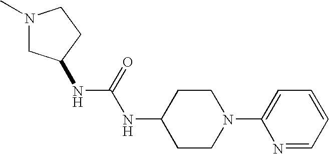 | 806.1 |
| 29 | 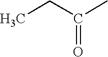 | 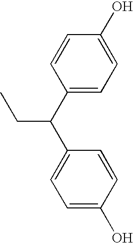 | 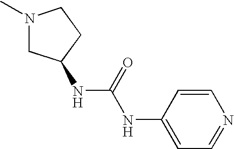 | 732.4 |
| 30 | 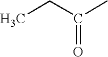 | 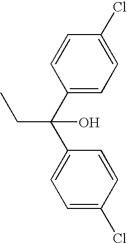 | 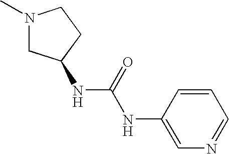 | 775.4 |
| 31 | 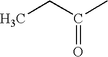 | 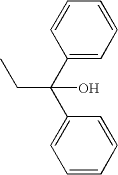 | 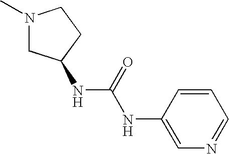 | 707.5 |
| 32 | 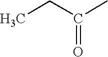 | 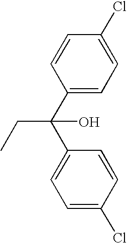 | 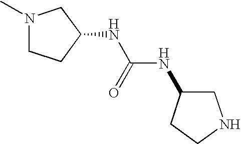 | 767.39 |
| 33 | 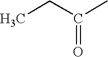 | 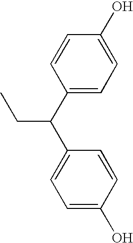 | —Cl | 553.2 |
| 34 | 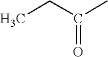 | 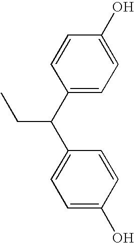 | 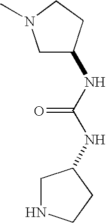 | 715.3 |
| 35 | 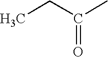 | 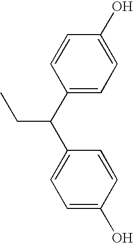 | 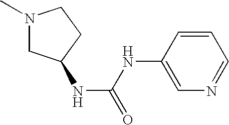 | 723.1 |
| 36 | 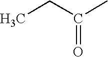 | 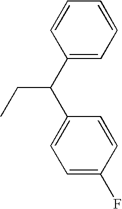 | 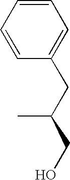 | 654.5 |
| 37 | 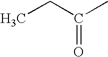 | 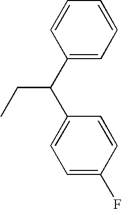 | 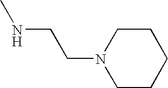 | 631.5 |
| 38 | 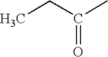 | 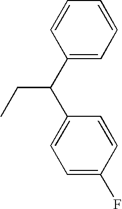 | 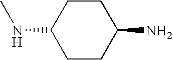 | 617.5 |
| 39 | 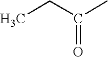 | 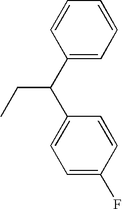 | 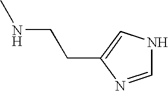 | 614.5 |
| 40 | 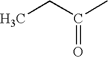 | 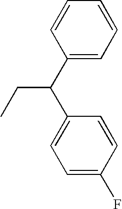 | 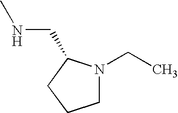 | 631.5 |
| 41 | 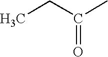 | 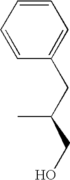 | 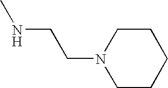 | 567.5 |
| 42 | 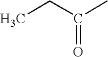 | 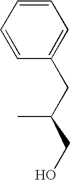 | 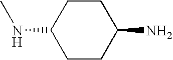 | 553.5 |
| 43 | 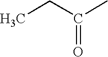 | 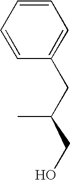 | 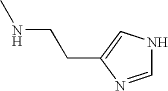 | 550.5 |
| 44 | 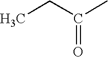 | 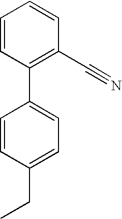 | 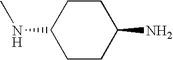 | 610.5 |
| 45 | 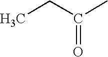 | 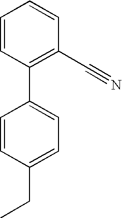 | 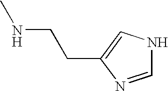 | 607.5 |
| 46 | 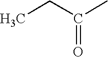 | 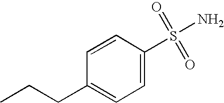 | 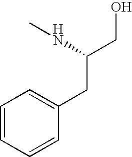 | 639.5 |
| 47 | 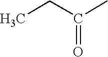 | 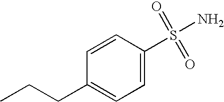 | 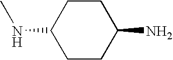 | 602.5 |
| 48 | 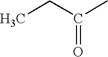 | 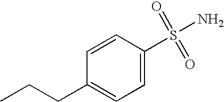 | 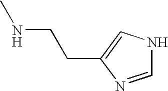 | 599.4 |
| 49 | 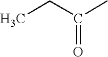 | 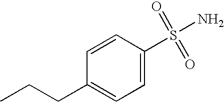 | 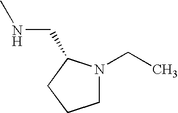 | 616.3 |
| 50 | 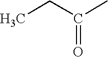 | 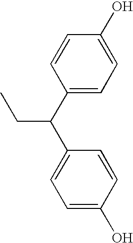 | 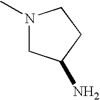 | 603.0 |

### Preparation of Intermediate Compounds

Abbreviations used are as follows: CDI is 1,1′-carbonyldiimidazole, DCM is dichloromethane, DIPEA is diisopropylethylamine, DMAP is 4-dimethylaminopyridine, DMF is dimethyl-formamide, DMSO is dimethylsulfoxide, LCMS is liquid chromatographic mass spectroscopy, TEA is triethylamine, TFA is trifluoroacetic acid, THF is tetrahydrofuran, EtOH is ethanol, IPA is isopropylalcohol and TLC is thin-layer chromatography.

### Intermediate A

### 2-Biphenyl-2-yl-ethylamine

2-Biphenylacetonitrile (1.00 g, 5.18 mmol) in dry THF (15 ml) under an atmosphere of Argon is treated with 1M borane-THF-complex (12.95 ml, 12.95 mmol) and heated at reflux for 6 hours. After cooling to room temperature, the solvent is removed in vacuo. The residue is suspended in MeOH (20 ml) and treated with conc. HCl (2 ml). The mixture is heated to reflux for 2 hours and after cooling to room temperature, the solvent is removed in vacuo. The residue is partitioned between DCM and water. The aqueous portion is treated with 4M NaOH and then extracted with DCM. The organic layer is dried (MgSO₄) and concentrated in vacuo to afford the title compound.

### Intermediate B

### 1,3-Di(R)-pyrrolidin-3-yl-urea

### B1: 1,3-Bis-((R)-1-benzyl-pyrrolidin-3-yl)-urea

A solution comprising (R)-1-benzyl-pyrrolidin-3-ylamine (5.0 g, 28.4 mmol) in DCM (10 ml) is treated with CDI (2.3 g, 14.2 mmol) and the reaction mixture is stirred at room temperature for 48 hours. The solvent is removed in vacuo and the resulting residue is dissolved in ethyl acetate. This portion is washed with water followed by brine, dried (MgSO₄) and concentrated in vacuo to yield the title compound as a pale orange solid.

### B2: 1,3-Di(R)pyrrolidin-3-yl-urea

To a solution of 1,3-bis-((R)-1-benzyl-pyrrolidin-3-yl)-urea (5.34 g, 14.1 mmol) in ethanol (80 ml) under an inert atmosphere of Argon is added palladium hydroxide on carbon (1.07 g). The reaction mixture is purged with Argon and placed under an atmosphere of hydrogen for two days after which time, the mixture is filtered and the catalyst washed with ethanol. The organic portions are combined and concentrated in vacuo to yield the title compound as a white solid.

### Intermediate C

### Imidazole-1-carboxylic acid (3,4,5,6-tetrahydro-2H-[1,2′]bipyridinyl-4-yl)-amide

A stirred solution of CDI (1.1 g, 6.77 mmol) in DCM (100 ml) is treated with 3,4,5,6-tetrahydro-2H-[1,2′]bipyridinyl-4-ylamine (WO 9965895 EP 21973) (1.0 g, 5.64 mmol in 50 ml of DCM) added dropwise over 30 minutes. The reaction mixture is stirred at room temperature for 15 minutes to yield the title compound as a 10 mg/ml solution in DCM. The compound is used in solution in subsequent reactions. This solution consists of the imidazole-urea intermediate (C) together with variable amounts of the corresponding isocyanate and imidazole.

### Intermediate D

### 2-Amino-1,1-bis-(4-chloro-phenyl)-ethanol

A reaction mixture comprising 4,4′-dichlorobenzophenone (5 g, 20 mmol) and zinc iodide (0.48 g, 1.49 mmol) in DCM (100 ml) is treated with cyanotrimethlysilane (2.17 g, 21.9 mmol) and stirred at room temperature overnight. The reaction mixture is diluted with water (100 ml) and the organic portion is separated, dried (MgSO₄) and concentrated in vacuo. The resulting residue is dissolved in THF, placed under an inert atmosphere of Argon and treated with 1M borane-THF-complex (40 ml, 40 mmol). The reaction mixture is then heated at reflux overnight and then concentrated in vacuo. The crude residue is treated carefully with MeOH (100 ml) followed by conc. HCl (5 ml). The reaction mixture is heated at reflux for 2 hours and then concentrated in vacuo to afford the title compound. (MH+ 282.09).

### Intermediate E

### 4,4′-(2-aminoethylidene)bis-phenol

This compound is prepared by the procedure of R. M. Schelkun et al. Bioorg. Med. Chem. Lett. 9 (1999) 2447-2452.

### Intermediate G

### [(1S,2R,3S,4R)-4-(2,6-dichloro-purin-9-yl)-2,3-dihydroxy-cyclopentyl]-propionyl-carbamic acid tert-butyl ester

### G1: (1S,4R)-4-(2,6-Dichloro-purin-9-yl)-cyclopent-2-enol

2,6-Dichloropurine (10 g, 52.90 mmol), (1S,4R)-cis 4-acetoxy-2-cyclopenten-1-ol (10 g. 70.40 mmol), tris(dibenzylideneacetone)dipalladium(0) (3.20 g, 3.50 mmol) and polymer supported triphenylphosphine (3 mmol/g, 11.60 g, 35.00 mmol) are placed in an oven-dried flask under an atmosphere of argon. Dry deoxygenated THF (80 ml) is added and the reaction mixture is stirred gently for 5 minutes. Triethylamine (20 ml) is added and the reaction mixture is stirred at 50° C. The reaction is shown to be complete by LCMS after 1 hour. The reaction mixture is allowed to cool, filtered and the solvent is removed in vacuo. The title compound is obtained after purification by flash column chromatography (silica, dichloromethane/methanol 25:1). ¹H nmr (CDCl₃, 400 MHz); 8.30 (s, 1H), 6.40 (m, 1H), 5.90 (m, 1H), 5.50 (m, 1H), 4.95 (m, 1H), 3.05 (m, 1H), 2.10 (m, 1H), MS (ES+) m/e 271 (MH⁺).

### G2: Carbonic acid (1S,4R)-4-(2,6-dichloro-purin-9-yl)-cyclopent-2-enyl ester ethyl ester

(1S,4R)-4-(2,6-Dichloro-purin-9-yl)-cyclopent-2-enol (9.5 g, 35.05 mmol) is placed in an oven-dried flask under an atmosphere of argon. Dry THF (200 mL) is added followed by dry pyridine (5.54 g, 70.1 mmol). Ethyl chloroformate (15.21 g, 140.2 mmol) is added slowly so that the temperature does not rise above 40° C. and the reaction mixture is stirred at room temperature. The reaction is shown to be complete by LCMS after 1 hour. The solvent is removed in vacuo and the residue is partitioned between dichloromethane (200 mL) and water (200 mL). The organic layer is washed with water (150 ml) and brine (150 ml), dried over MgSO₄, filtered and the solvent is removed in vacuo. The title compound is obtained after crystallisation from methanol. ¹H nmr (CDCl₃, 400 MHz); 8.20 (s, 1H), 6.45 (m, 1H), 6.25 (m, 1H), 5.75 (m, 1H), 5.70 (m, 1H), 4.25 (q, 2H), 3.20 (m, 1H), 2.05 (m, 1H), 1.35 (t, 3H), MS (ES+) m/e 343 (MH⁺).

### G3: [(1S,4R)-4-(2,6-Dichloro-purin-9-yl)-cyclopent-2-enyl]-propionyl-carbamic acid tert-butyl ester

Carbonic acid (1S,4R)-4-(2,6-dichloro-purin-9-yl)-cyclopent-2-enyl ester ethyl ester (1.00 g, 2.92 mmol), propionyl-carbamic acid tert-butyl ester (Intermediate W) (0.55 g, 3.21 mmol) and triphenylphosphine (0.115 g, 0.44 mmol) are placed under an inert atmosphere of Argon. THF (10 ml) is added followed by tris(dibenzylideneacetone)dipalladium(0) (0.13 g, 0.15 mmol). The reaction mixture is stirred at 50° for 1 hour. The solvent is removed in vacuo and purification by chromatography on silica eluting with EtOAc/hexane (1:4) affords the title product. ¹H nmr (CDCl₃, 400 MHz); 8.70 (s, 1H), 6.15 (m, 1H), 5.85 (m, 1H), 5.80 (m, 1H), 5.60 (m, 1H), 3.15 (m, 1H), 2.75 (q, 2H), 2.10 (m, 1H), 1.55 (s, 9H), 1.15 (t, 3H), MS (ES+) m/e 426 (MH⁺).

### G4: [(1S,2R,3S,4R)-4-(2,6-Dichloro-purin-9-yl)-2,3-dihydroxy-cyclopentyl]-propionyl-carbamic acid tert-butyl ester

[(1S,4R)-4-(2,6-dichloro-purin-9-yl)-cyclopent-2-enyl]-propionyl-carbamic acid tert-butyl ester (11.37 g, 26.7 mmol), methanesulfonamide (2.54 g, 26.7 mmol) and AD-mix-a (55 g) are placed in a flash of water (100 ml) and t-butanol (100 ml). Osmium tetroxide (4% in water) is added and the reaction mixture is stirred vigorously at room temperature overnight. Sodium sulfite (40 g) is added and the mixture is stirred at room temperature for a further hour and then partitioned between EtOAc and water. The organic portion is separated, dried (MgSO₄) and concentrated in vacuo. The crude product is purified by chromatography on silica eluting with DCM:MeOH (25:1 increasing to 10:1) to afford the title compound.

### Intermediate T

### 2-Amino-1,1-bis-(4-fluoro-phenyl)-ethanol

A solution of 2-benzylamino-1,1-bis-(4-fluoro-phenyl)-ethanol (240 mg) in EtOH (30 ml) is treated with 20% Pd/C (50 mg) and hydrogenated at 0.35 bar for 2 days. The reaction mixture is concentrated in vacuo and the residues partitioned between ethyl acetate/water. The organic portion is separated, dried (Na₂SO₄) and concentrated in vacuo to afford the titled compound (168 mg).

### Intermediate V

### 2-(1-Ethyl-1H-imidazol-4-yl)-ethylamine

This compound is prepared by the procedure of Rahul Jain and Louis A. Cohen Tetrahedron 1996, 52, 5363.

### Intermediate W

### Propionyl-carbamic acid tert-butyl ester

The title compound is prepared from propyl-carbamic acid tert-butyl ester using the procedure described by Ken-ichi Takana et al in Chem. Pharm. Bull. 1988, 36, 3125. ¹H nmr (CDCl₃, 400 MHz); 7.25 (br s, 1H), 2.75 (q, 2H), 1.50 (s, 9H), 1.15 (t, 3H).

### Intermediate ZB

### Pyridin-3-yl-carbamic acid phenyl ester

A solution of pyridine (2 ml) in DCM (10 ml) is treated with phenylchloroformate (1.83 g, 11.7 mmol). To this solution is added 3-aminopyridine (1.0 g, 10.6 mmol) in DCM (8 ml) which results in an exotherm of 20° C. The reaction mixture is stirred at room temperature for 2 hours and then concentrated in vacuo. The residue is partitioned between EtOAc and water and the organic portion is separated. This organic portion is washed with water, saturated sodium bicarbonate solution, dried (MgSO₄) and concentrated in vacuo to afford the title compound as a white solid. (MH+215.13)

### PREPARATION OF SPECIFIC EXAMPLES

### Example 1

### N-((1S,2R,3S,4R)-4-{2-Chloro-6-[(9H-fluoren-9-ylmethyl)-amino]-purin-9-yl}-2,3-dihydroxy-cyclopentyl)-propionamide trifluoroacetate

A solution comprising [(1S,2R,3S,4R)-4-(2,6-dichloro-purin-9-yl)-2,3-dihydroxy-cyclopentyl]-propionyl-carbamic acid tert-butyl ester (Intermediate G) (0.5 g, 1.1 mmol), DIPEA (0.227 ml, 1.3 mmol), fluoren-9-yl-methylamine hydrochloride (256 mg, 1.2 mmol) in 1,2-dichloroethane (3 ml) is heated at 50° C. overnight. Hydrochloric acid (10 ml of a 0.1 M solution) is added to the reaction mixture and following agitation, the organic portion is separated and treated with TFA (1 ml). After standing at room temperature for 2 hours, the solvent is removed in vacuo to yield the title compound.

### Examples 2-3

These compounds,
N-{(1S,2R,3S,4R)-4-[2-chloro-6-((S)-1-hydroxymethyl-2-phenyl-ethylamino)-purin-9-yl]-2,3-dihydroxy-cyclopentyl}-propionamide trifluoroacetate (Example 2) and
N-((1S,2R,3S,4R)-4-{2-chloro-6-[2-(4-sulfamoyl-phenyl)-ethylamino]-purin-9-yl}-2,3-dihydroxy-cyclopentyl)-propionamide trifluoroacetate (Example 3)
   are prepared analogously to N-((1S,2R,3S,4R)-4-{2-chloro-6-[(9H-fluoren-9-ylmethyl)-amino]-purin-9-yl}-2,3-dihydroxy-cyclopentyl)-propionamide trifluoroacetate (Example 1) by replacing fluoren-9-yl-methylamine hydrochloride with the appropriate amine.

### Examples 4-6

These compounds, namely
N-{(1S,2R,3S,4R)-4-[6-(2-biphenyl-2-yl-ethylamino)-2-chloro-purin-9-yl]-2,3-dihydroxy-cyclopentyl}-propionamide (Example 4),
N-((1S,2R,3S,4R)-4-{6-[2,2-bis-(3,4-dimethoxy-phenyl)-ethylamino]-2-chloro-purin-9-yl}-2,3-dihydroxy-cyclopentyl)-propionamide (Example 5) and
N-((1S,2R,3S,4R)-4-{2-chloro-6-[2-(4-methoxy-phenyl)-2-phenyl-ethylamino]-purin-9-yl}-2,3-dihydroxy-cyclopentyl)-propionamide (Example 6) are prepared analogously to N-((1S,2R,3S,4R)-4-{2-chloro-6-[(9H-fluoren-9-ylmethyl)-amino]-purin-9-yl}-2,3-dihydroxy-cyclopentyl)-propionamide trifluoroacetate (Example 1) by replacing fluoren-9-yl-methylamine hydrochloride with the appropriate amine. These examples are also treated with potassium carbonate/methanol to afford the product in free form.

### Example 7

### N-((1S,2R,3S,4R)-4-{6-[2,2-Bis-(4-methoxy-phenyl)-ethylamino]-2-[(R)-3-((R)-3-pyrrolidin-3-ylureido)-pyrrolidin-1-yl]-purin-9-yl}-2,3-dihydroxy-cyclopentyl)-propionamide trifluoroacetate

A solution comprising N-((1S,2R,3S,4R)-4-{6-[2,2-Bis-(4-methoxy-phenyl)-ethylamino]-2-chloro-purin-9-yl}-2,3-dihydroxy-cyclopentyl)-propionamide (prepared from [(1S,2R,3S,4R)-4-(2,6-dichloro-purin-9-yl)-2,3-dihydroxy-cyclopentyl]-propionyl-carbamic acid tert-butyl ester (Intermediate G) analogously to Example 1 with the appropriate amine) (0.02 g, 0.03 mmol) and 1,3-di(R)-pyrrolidin-3-yl-urea (Intermediate B) in DMSO (0.2 ml) is heated to 100° C. for 24 hours. Purification is carried out using mass directed preparative LC-MS eluting with acetonitrile:water:trifluoroacetic acid to afford the title compound.

### Example 8-11

These compounds, namely
N-((1S,2R,3S,4R)-4-{6-[2-(4-fluoro-phenyl)-2-phenyl-ethylamino]-2-[(R)-3-((R)-3-pyrrolidin-3-ylureido)-pyrrolidin-1-yl]-purin-9-yl}-2,3-dihydroxy-cyclopentyl)-propionamide trifluoroacetate (Example 8),
N-((1S,2R,3S,4R)-4-{6-[(9H-fluoren-9-ylmethyl)-amino]-2-[(R)-3-((R)-3-pyrrolidin-3-ylureido)-pyrrolidin-1-yl]-purin-9-yl}-2,3-dihydroxy-cyclopentyl)-propionamide trifluoroacetate (Example 9),
N-((1S,2R,3S,4R)-2,3-dihydroxy-4-{6-((S)-1-hydroxymethyl-2-phenyl-ethylamino)-2-[(R)-3-((R)-3-pyrrolidin-3-ylureido)-pyrrolidin-1-yl]-purin-9-yl}-cyclopentyl)-propionamide trifluoroacetate (Example 10) and
N-((1S,2R,3S,4R)-4-{6-[(2′-cyano-biphenyl-4-ylmethyl)-amino]-2-[(R)-3-((R)-3-pyrrolidin-3-ylureido)-pyrrolidin-1-yl]purin-9-yl}-2,3-dihydroxy-cyclopentyl)-propionamide trifluoroacetate (Example 11) are prepared analogously to Example 7 by replacing N-((1S,2R,3S,4R)-4-{6-[2,2-Bis-(4-methoxy-phenyl)-ethylamino]-2-chloro-purin-9-yl}-2,3-dihydroxy-cyclopentyl)-propionamide with the appropriate starting compound the preparation of which is described herein. The synthesis of the starting compound used for the preparation of Example 11 is not described but it is synthesised using an analogous procedure to Example 1 with the appropriate amine.

### Example 12

### N-((1S,2R,3S,4R)-4-{2-((R)-3-Amino-pyrrolidin-1-yl)-6-[2,2-bis-(4-methoxy-phenyl)-ethylamino]-purin-9-yl}-2,3-dihydroxy-cyclopentyl)-propionamide trifluoroacetate

A mixture comprising N-((1S,2R,3S,4R)-4-{6-[2,2-bis-(4-methoxy-phenyl)-ethylamino]-2-chloro-purin-9-yl}-2,3-dihydroxy-cyclopentyl)-propionamide (prepared analogously to Example 1 with the appropriate amine) (0.02 g, 0.03 mmol), (3R)-3-(Boc-amino)pyrrolidine (28 mg, 0.15 mmol), sodium iodide (4 mg, 0.03 mmol) in NMP/acetonitrile (0.5 ml of a 1:1 mixture) is heated using microwave radiation in a Personal Chemistry Emrys™ Optimizer microwave reactor at 160° C. for 30 minutes. DCM (3 ml) and water (3 ml) are added to the reaction mixture and following agitation, the organic portion is separated and treated with TFA (0.5 ml). After standing at room temperature overnight purification is carried out using mass directed preparative LC-MS eluting with acetonitrile:water:trifluoroacetic acid to afford the title compound.

### Examples 13-16

These compounds, namely,
N-((1S,2R,3S,4R)-4-{2-((R)-3-amino-pyrrolidin-1-yl)-6-[2-(4-fluoro-phenyl)-2-phenyl-ethylamino]-purin-9-yl}-2,3-dihydroxy-cyclopentyl)-propionamide trifluoroacetate (Example 13),
N-((1S,2R,3S,4R)-4-{2-((R)-3-amino-pyrrolidin-1-yl)-6-[(9H-fluoren-9-ylmethyl)-amino]-purin-9-yl}-2,3-dihydroxy-cyclopentyl)-propionamide trifluoroacetate (Example 14),
N-((1S,2R,3S,4R)-4-{2-((R)-3-amino-pyrrolidin-1-yl)-6-[(2′-cyano-biphenyl-4-ylmethyl)-amino]-purin-9-yl}-2,3-dihydroxy-cyclopentyl)-propionamide trifluoroacetate (Example 15) and
N-((1S,2R,3S,4R)-4-{2-((R)-3-amino-pyrrolidin-1-yl)-6-[2-(4-sulfamoyl-phenyl)-ethylamino]-purin-9-yl}-2,3-dihydroxy-cyclopentyl)-propionamide trifluoroacetate (Example 16)
   are prepared analogously to Example 12 by replacing N-((1S,2R,3S,4R)-4-{6-[2,2-bis-(4-methoxy-phenyl)-ethylamino]-2-chloro-purin-9-yl}-2,3-dihydroxy-cyclopentyl)-propionamide with the appropriate starting compound, the preparation of which is described herein.

### Examples 17-20

These compounds namely,
N-((1S,2R,3S,4R)-4-{6-(2-biphenyl-2-yl-ethylamino)-2-[(R)-3-((R)-3-pyrrolidin-3-ylureido)-pyrrolidin-1-yl]-purin-9-yl}-2,3-dihydroxy-cyclopentyl)-propionamide trifluoroacetate (Example 17),
N-((1S,2R,3S,4R)-4-{6-[2,2-bis-(4-chloro-phenyl)-ethylamino]-2-[(R)-3-((R-3-pyrrolidin-3-ylureido)-pyrrolidin-1-yl]-purin-9-yl}-2,3-dihydroxy-cyclopentyl)-propionamide trifluoroacetate (Example 18),
N-((1S,2R,3S,4R)-4-{6-[2,2-bis-(3,4-dimethoxy-phenyl)-ethylamino]-2-[(R)-3-((R)-3-pyrrolidin-3-ylureido)-pyrrolidin-1-yl]-purin-9-yl}-2,3-dihydroxy-cyclopentyl)-propionamide trifluoroacetate (Example 19) and
N-((1S,2R,3S,4R)-2,3-dihydroxy-4-{6-[2-(4-methoxy-phenyl)-2-phenyl-ethylamino]-2-[(R)-3-((R)-3-pyrrolidin-3-ylureido)-pyrrolidin-1-yl]-purin-9-yl}-cyclopentyl)-propionamide trifluoroacetate (Example 20)
   are prepared analogously to Example 7 by replacing N-((1S,2R,3S,4R)-4-{6-[2,2-Bis-(4-methoxy-phenyl)-ethylamino]-2-chloro-purin-9-yl}-2,3-dihydroxy-cyclopentyl)-propionamide with the appropriate starting compound, the preparations of which are described herein. The synthesis of the starting compound used for the preparation of Examples 18 is not described but it is synthesised using an analogous procedure to Example 1 with the appropriate amine.

### Examples 21-25

These compounds namely,
N-((1S,2R,3S,4R)-4-{6-(2-biphenyl-2-yl-ethylamino)-2-[2-(1-ethyl-1H-imidazol-4-yl)-ethylamino]-purin-9-yl}-2,3-dihydroxy-cyclopentyl)-propionamide trifluoroacetate (Example 21),
N-((1S,2R,3S,4R)-4-{6-[2,2-bis-(4-chloro-phenyl)-2-hydroxy-ethylamino]-2-[2-(1-ethyl-1H-imidazol-4-yl)-ethylamino]-purin-9-yl}-2,3-dihydroxy-cyclopentyl)-propionamide trifluoroacetate (Example 22),
N-((1S,2R,3S,4R)-4-{6-[2,2-bis-(4-chloro-phenyl)-ethylamino]-2-[2-(1-ethyl-1H-imidazol-4-yl)-ethylamino]-purin-9-yl}-2,3-dihydroxy-cyclopentyl)-propionamide trifluoroacetate (Example 23),
N-((1S,2R,3S,4R)-4-{6-[2,2-bis-(3,4-dimethoxy-phenyl)-ethylamino]-2-[2-(1-ethyl-1H-imidazol-4-yl)-ethylamino]-purin-9-yl}-2,3-dihydroxy-cyclopentyl)-propionamide trifluoroacetate (Example 24) and
N-((1S,2R,3S,4R)-4-{2-[2-(1-ethyl-1H-imidazol-4-yl)-ethylamino]-6-[2-(4-methoxy-phenyl)-2-phenyl-ethylamino]-purin-9-yl}-2,3-dihydroxy-cyclopentyl)-propionamide trifluoroacetate (Example 25)
   are prepared analogously to Example 12 by replacing (3R)-3-Boc-amino)pyrrolidine with 2-(1-ethyl-1H-imidazol-4-yl)-ethylamine and by replacing N-((1S,2R,3S,4R)-4-{6-[2,2-bis-(4-methoxy-phenyl)-ethylamino]-2-chloro-purin-9-yl}-2,3-dihydroxy-cyclopentyl)-propionamide with the appropriate starting materials, the preparations of which are described herein. The synthesis of the starting compounds used for the preparation of Examples 22 and 23 are not described but are synthesised using an analogous procedure to Example 1 with the appropriate amine.

### Example 26

### N-((1S,2R,3S,4R)-2,3-Dihydroxy-4-{2-[(R)-3-((R)-3-pyrrolidin-3-ylureido)-pyrrolidin-1-yl]-6-[2-(4-sulfamoyl-phenyl)-ethylamino]-purin-9-yl}-cyclopentyl)-propionamide trifluoroacetate

This compound is prepared analogously to N-((1S,2R,3S,4R)-4-{6-[2,2-Bis-4-methoxy-phenyl)-ethylamino]-2-[(R)-3-((R)-3-pyrrolidin-3-ylureido)-pyrrolidin-1-yl]-purin-9-yl}-2,3-dihydroxy-cyclopentyl)-propionamide trifluoroacetate (Example 7) by replacing N-((1S,2R,3S,4R)-4-{6-[2,2-Bis-(4-methoxy-phenyl)-ethylamino]-2-chloro-purin-9-yl}-2,3-dihydroxy-cyclopentyl)-propionamide with N-((1S,2R,3S,4R)-4-{2-chloro-6-[2-(4-sulfamoyl-phenyl)-ethylamino]-purin-9-yl}-2,3-dihydroxy-cyclopentyl)-propionamide trifluoroacetate (Example 3).

### Examples 27

### N-((1S,2R,3S,4R)-4-{2-Chloro-6-[2-(4-fluoro-phenyl)-2-phenyl-ethylamino]-purin-9-yl}-2,3-dihydroxy-cyclopentyl)-propionamide trifluoroacetate

A solution comprising [(1S,2R,3S,4R)-4-(2,6-dichloro-purin-9-yl)-2,3-dihydroxy-cyclopentyl]-propionyl-carbamic acid tert-butyl ester (Intermediate G) (0.5 g, 1.1 mmol), DIPEA (0.227 ml, 1.3 mmol), 2-(4-fluorophenyl)-2-phenylethylamine hydrochloride (257 mg, 1.2 mmol) in 1,2-dichloroethane (3 ml) is heated at 50° C. overnight. Hydrochloric acid (10 ml of a 0.1 M solution) is added to the reaction mixture and following agitation, the organic portion is separated and treated with TFA (1 ml). After standing at room temperature for 2 hours, the solvent is removed in vacuo to yield the title compound.

### Example 28

### N-[(1S,2R,3S,4R)-4-(6-[2,2-Bis-(4-hydroxy-phenyl)-ethylamino]-2-{(R)-3-[3-(3,4,5,6-tetrahydro-2H-[1,2′]bipyridinyl-4-yl)ureido]-pyrrolidin-1-yl}-purin-9-yl)-2,3-dihydroxy-cyclopentyl]-propionamide

A suspension of N-((1S,2R,3S,4R)-4-{2-((R)-3-amino-pyrrolidin-1-yl)-6-[2,2-bis-(4-hydroxy-phenyl)-ethylamino]-purin-9-yl}-2,3-dihydroxy-cyclopentyl)-propionamide trifluoroacetate (Example 50) (360 mg, 0.5 mmol) in IPA (3 ml) is treated with imidazole-1-carboxylic acid (3,4,5,6-tetrahydro-2H-[1,2′]bipyridinyl-4-yl)amide (Intermediate C) (16.2 ml of a 10 mg/ml solution in DCM, 0.6 mmol) and the reaction mixture is stirred at room temperature overnight. The solvent is removed in vacuo and purification by reverse phase column chromatography (Isolute™ C18, 10-50% acetonitrile in water—0.1% TFA) affords the product which is further purified by treating with potassium carbonate and passing through a pre-washed (400 ml MeOH followed by 400 ml water) eluting with 0.5% ammonia 880:water followed by water and finally MeOH to afford the title compound.

### Example 29

### N-((1S,2R,3S,4R)-4-{6-[2,2-Bis-(4-hydroxy-phenyl)-ethylamino]-2-[(R)-3-(3-pyridin-4-yl-ureido)-pyrrolidin-1-yl]-purin-9-yl}-2,3-dihydroxy-cyclopentyl)-propionamide hydrochloride

A mixture comprising N-((1S,2R,3S,4R)-4-{2-((R)-3-amino-pyrrolidin-1-yl)-6-[2,2-bis-(4-hydroxy-phenyl)-ethylamino]-purin-9-yl}-2,3-dihydroxy-cyclopentyl)-propionamide trifluoroacetate (Example 50) (20 mg, 33 μmol) and 4-pyridinecarbamic acid phenyl ester (7.8 mg, 37 μmol) in NMP (0.5 ml) is stirred and heated to 100° C. for 1 hour and then allowed to cool to room temperature overnight. Purification by C-18 reverse phase column chromatography eluting with acetonitrile:water:HCl (0.1%) (gradient of 0 to 100% acetonitrile) yields the title compound.

### Example 30

### N-((1S,2R,3S,4R)-4-{6-[2,2-Bis-(4-chloro-phenyl)-2-hydroxy-ethylamino]-2-[(R)-3-(3-pyridin-3-yl-ureido)-pyrrolidin-1-yl]-purin-9-yl}-2,3-dihydroxy-cyclopentyl)-propionamide hydrochloride

### Step 1: {(R)-1-[6-[2,2-Bis-(4-chloro-phenyl)-2-hydroxy-ethylamino]-9-((1R,2S,3R,4S)-2,3-dihydroxy-4-propionylamino-cyclopentyl)-9H-purin-2-yl]-pyrrolidin-3-yl}-carbamic acid tert-butyl ester

N-((1S,2R,3S,4R)-4-{6-[2,2-Bis-(4-chloro-phenyl)-2-hydroxy-ethylamino]-2-chloro-purin-9-yl}-2,3-dihydroxy-cyclopentyl)-propionamide (prepared analogously to Example 1 with the appropriate amine) (0.2 g, 0.33 mmol), (R)-3-(Boc-amino)pyrrolidine (0.186 g, 1 mmol) and sodium iodide (0.05 g, 0.33 mmol) in acetonitrile (2 ml) are heated using microwave radiation in a Personal Chemistry Emrys™ Optimizer microwave reactor at 160° C. for 1 hour. The solvent is removed in vacuo and the residue is dissolved in DCM (30 ml) and washed with 0.1 M HCl (25 ml), water (25 ml), brine (5 ml), dried (MgSO4) and concentrated in vacuo to afford the title compound which is used without further purification. MS (ES+) m/e 755 (MH⁺).

### Step 2: N-((1S,2R,3S,4R)-4-{2-((R)-3-Amino-pyrrolidin-1-yl)-6-[2,2-bis-(4-chloro-phenyl)-2-hydroxy-ethylamino]-purin-9-yl}-2,3-dihydroxy-cyclopentyl)-propionamide

A solution of {(R)-1-[6-[2,2-Bis-(4-chloro-phenyl)-2-hydroxy-ethylamino]-9-((1R,2S,3R,4S)-2,3-dihydroxy-4-propionylamino-cyclopentyl)-9H-purin-2-yl]-pyrrolidin-3-yl}-carbamic acid tert-butyl ester (0.2 g, 0.26 mmol) in MeOH (2 ml) is treated with 4M HCl in dioxane (2 ml) and stirred at room temperature. The solvent is removed in vacuo to afford the title compound as a yellow foam. MS (ES+) m/e 655 (MH⁺).

### Step 3: N-((1S,2R,3S,4R)-4-{6-[2,2-Bis-(4-chloro-phenyl)-2-hydroxy-ethylamino]-2-[(R)-3-(3-pyridin-3-yl-ureido)-pyrrolidin-1-yl]-purin-9-yl}-2,3-dihydroxy-cyclopentyl)-propionamide hydrochloride

A reaction mixture comprising N-((1S,2R,3S,4R)-4-{2-((R)-3-Amino-pyrrolidin-1-yl)-6-[2,2-bis-(4-chloro-phenyl)-2-hydroxy-ethylamino]-purin-9-yl}-2,3-dihydroxy-cyclopentyl)-propionamide (170 mg, 0.26 mmol) and TEA (42 μl, 0.3 mmol) in THF (10 ml) and DMF (1 ml) is treated with 3-pyridyl isocyanate (36 mg, 0.3 mmoL) and stirred at room temperature for 1 hour. The reaction mixture is diluted with MeOH/EtOAc (100 ml of a 1:9 mixture) and washed consecutively with 0.1 M HCl (50 ml), water (50 ml) and brine (20 ml). The mixture is dried (MgSO₄) and concentrated in vacuo. The crude product is purified by C-18 reverse phase column chromatography eluting with acetonitrile:water:HCl (0.1%) (gradient of 0 to 100% acetonitrile) yields the title compound.

### Example 31

### N-((1S,2R,3S,4R)-2,3-Dihydroxy-4-{6-(2-hydroxy-2,2-diphenyl-ethylamino)-2-[(R)-3-(3-pyridin-3-ylureido)-pyrrolidin-1-yl]-purin-9-yl}-cyclopentyl)-propionamide

A solution of N-((1S,2R,3S,4R)-4-{6-[2,2-Bis-(4-chloro-phenyl)-2-hydroxy-ethylamino]-2-[(R)-3-(3-pyridin-3-yl-ureido)-pyrrolidin-1-yl]purin-9-yl}-2,3-dihydroxy-cyclopentyl)-propionamide hydrochloride (Example 30) (30 mg, 0.039 mmol) in ethanol (2 ml) under an inert atmosphere of Argon is added 10% palladium hydroxide on carbon (0.04 g) followed by ammonium formate (0.025 g, 0.39 mmol). The reaction mixture is purged with Argon, heated to reflux for 1 hour and then allowed to cool to room temperature. The mixture is filtered through Celite® and the catalyst washed with ethanol. The organic portions are combined and concentrated in vacuo to yield the title compound as a colourless solid. MS (ES+) m/e 708 (MH⁺).

### Example 32

### N-((1S,2R,3S,4R)-4-{6-[2,2-Bis-(4-chloro-phenyl)-2-hydroxy-ethylamino]-2-[(R)-3-((R)-3-pyrrolidin-3-ylureido)-pyrrolidin-1-yl]-purin-9-yl}-2,3-dihydroxy-cyclopentyl)-propionamide trifluoroacetate

This compound is prepared analogously to Example 7 by replacing N-((1S,2R,3S,4R)-4-{6-[2,2-Bis-(4-methoxy-phenyl)-ethylamino]-2-chloro-purin-9-yl}-2,3-dihydroxy-cyclopentyl)-propionamide with N-((1S,2R,3S,4R)-4-{6-[2,2-bis-(4-chloro-phenyl)-2-hydroxy-ethylamino]-2-chloro-purin-9-yl}-2,3-dihydroxy-cyclopentyl)-propionamide [prepared analogously to Example 1 replacing fluoren-9-yl-methylamine hydrochloride with 2-amino-1,1-bis-(4-chloro-phenyl)-ethanol (Intermediate D)].

### Example 33

### N-((1S,2R,3S,4R)-4-{6-[2,2-Bis-(4-hydroxy-phenyl)-ethylamino]-2-chloro-purin-9-yl}-2,3-dihydroxy-cyclopentyl)-propionamide

This compound is prepared analogously to N-((1S,2R,3S,4R)-4-{2-chloro-6-[2-(4-fluoro-phenyl)-2-phenyl-ethylamino]-purin-9-yl}-2,3-dihydroxy-cyclopentyl)-propionamide trifluoroacetate (Example 1) by replacing fluoren-9-yl-methylamine hydrochloride with 4,4′-(2-aminoethylidene)bis-phenol. The free form of the compound is obtained by partitioning the TFA salt between ethyl acetate and saturated sodium hydrogen carbonate solution. The organic portion is separated and washed with brine, dried (MgSO4) and concentrated in vacuo to afford the title compound in its free form.

### Example 34

### N-((1S,2R,3S,4R)-4-{6-[2,2-Bis-(4-hydroxy-phenyl)-ethylamino]-2-[(R)-3-((R)-3-pyrrolidin-3-ylureido)-pyrrolidin-1-yl]-purin-9-yl}-2,3-dihydroxy-cyclopentyl)-propionamide trifluoroacetate

This compound is prepared analogously to Example 7 by replacing N-((1S,2R,3S,4R)-4-{6-[2,2-Bis-(4-methoxy-phenyl)-ethylamino]-2-chloro-purin-9-yl}-2,3-dihydroxy-cyclopentyl)-propionamide with N-((1S,2R,3S,4R)-4-{6-[2,2-bis-(4-hydroxy-phenyl)-ethylamino]-2-chloro-purin-9-yl}-2,3-dihydroxy-cyclopentyl)-propionamide (Example 33).

### Example 35

### N-((1S,2R,3S,4R)-4-{6-[2,2-Bis-(4-hydroxy-phenyl)-ethylamino]-2-[(R)-3-(3-pyridin-3-yl-ureido)-pyrrolidin-1-yl]-purin-9-yl}-2,3-dihydroxy-cyclopentyl)-propionamide

A mixture comprising N-((1S,2R,3S,4R)-4-{2-((R)-3-amino-pyrrolidin-1-yl)-6-[2,2-bis-(4-hydroxy-phenyl)-ethylamino]-purin-9-yl}-2,3-dihydroxy-cyclopentyl)-propionamide trifluoroacetate (Example 69) (20 mg, 33 μmol) in THF (0.5 ml) and NMP (0.5 ml) is treated with TEA (13 mg, 0.13 mmol) followed by 3-pyridyl isocyanate (16 mg, 83 μmol). After stirring at room temperature for 2 hours, the solvent is removed in vacuo and purification by C-18 reverse phase column chromatography eluting with acetonitrile:water:TFA (0.1%) (gradient of 0 to 100% acetonitrile) yields the title compound. MS (ES+) m/e 708 (MH⁺).

### Example 36-40

These compounds namely,
N-{(1S,2R,3S,4R)-4-[6-[2-(4-fluoro-phenyl)-2-phenyl-ethylamino]-2-((S)-1-hydroxymethyl-2-phenyl-ethylamino)-purin-9-yl]-2,3-dihydroxy-cyclopentyl}-propionamide trifluoroacetate (Example 36),
N-{(1S,2R,3S,4R)-4-[6-[2-(4-fluoro-phenyl)-2-phenyl-ethylamino]-2-(2-piperidin-1-yl-ethylamino)-purin-9-yl]-2,3-dihydroxy-cyclopentyl}-propionamide trifluoroacetate (Example 37),
N-((1S,2R,3S,4R)-4-{2-(4-Amino-cyclohexylamino)-6-[2-(4-fluoro-phenyl)-2-phenyl-ethylamino]-purin-9-yl}-2,3-dihydroxy-cyclopentyl)-propionamide trifluoroacetate (Example 38),
N-((1S,2R,3S,4R)-4-{6-[2-(4-fluoro-phenyl)-2-phenyl-ethylamino]-2-[2-(1H-imidazol-4-yl)ethylamino]-purin-9-yl}-2,3-dihydroxy-cyclopentyl)-propionamide trifluoroacetate (Example 39),
N-((1S,2R,3S,4R)-4-{2-[((R)-1-ethyl-pyrrolidin-2-ylmethyl)-amino]-6-[2-(4-fluoro-phenyl)-2-phenyl-ethylamino]-purin-9-yl}-2,3-dihydroxy-cyclopentyl)-propionamide trifluoroacetate (Example 40) are prepared analogously to N-((1S,2R,3S,4R)-4-{2-((R)-3-amino-pyrrolidin-1-yl)-6-[2,2-bis-(4-methoxy-phenyl)-ethylamino]-purin-9-yl}-2,3-dihydroxy-cyclopentyl)-propionamide trifluoroacetate (Example 12) by replacing N-((1S,2R,3S,4R)-4-{6-[2,2-bis-(4-methoxy-phenyl)-ethylamino]-2-chloro-purin-9-yl}-2,3-dihydroxy-cyclopentyl)-propionamide with N-((1S,2R,3S,4R)-4-{2-chloro-6-[2-(4-fluoro-phenyl)-2-phenyl-ethylamino]-purin-9-yl}-2,3-dihydroxy-cyclopentyl)-propionamide trifluoroacetate (prepared analogously to Example 1 with the appropriate amine) and by replacing (3R)-3-(Boc-amino)pyrrolidine with the appropriate amine.

### Example 41-43

These compounds namely,
N-{(1S,2R,3S,4R)-4-[6-((S)-1-benzyl-2-hydroxy-ethylamino)-2-(2-piperidin-1-yl-ethylamino)-purin-9-yl]-2,3-dihydroxy-cyclopentyl}-propionamide trifluoroacetate (Example 41),
N-{(1S,2R,3S,4R)-4-[2-(4-amino-cyclohexylamino)-6-((S)-1-benzyl-2-hydroxy-ethylamino)-purin-9-yl]-2,3-dihydroxy-cyclopentyl}-propionamide trifluoroacetate (Example 42),
N-((1S,2R,3S,4R)-4-{6-((S)-1-benzyl-2-hydroxy-ethylamino)-2-[2-(1H-imidazol-4-yl)-ethylamino]-purin-9-yl}-2,3-dihydroxy-cyclopentyl)-propionamide trifluoroacetate (Example 43),
   are prepared analogously to N-((1S,2R,3S,4R)-4-{2-((R)-3-amino-pyrrolidin-1-yl)-6-[2,2-bis(4-methoxy-phenyl)-ethylamino]-purin-9-yl}-2,3-dihydroxy-cyclopentyl)-propionamide trifluoroacetate (Example 12) by replacing N-(1S,2R,3S,4R)-4-{6-[2,2-bis-4-methoxy-phenyl)-ethylamino]-2-chloro-purin-9-yl}-2,3-dihydroxy-cyclopentyl)-propionamide with N-{(1S,2R,3S,4R)-4-[2-chloro-6-((S)-1-hydroxymethyl-2-phenyl-ethylamino)-purin-9-yl]-2,3-dihydroxy-cyclopentyl}-propionamide trifluoroacetate (Example 2) and by replacing (3R)-3-(Boc-amino)pyrrolidine with the appropriate amine.

### Example 44-45

These compounds namely,
N-((1S,2R,3S,4R)-4-{2-(4-amino-cyclohexylamino)-6-[(2′-cyano-biphenyl-4-ylmethyl)-amino]-purin-9-yl}-2,3-dihydroxy-cyclopentyl)-propionamide trifluoroacetate (Example 44) and
N-((1S,2R,3S,4R)-4-{6-[(2′-cyano-biphenyl-4-ylmethyl)-amino]-2-[2-(1H-imidazol-4-yl)-ethylamino]-purin-9-yl}-2,3-dihydroxy-cyclopentyl)-propionamide trifluoroacetate (Example 45)
   are prepared analogously to N-((1S,2R,3S,4R)-4-{2-((R)-3-amino-pyrrolidin-1-yl)-6-[2,2-bis(4-methoxy-phenyl)-ethylamino]-purin-9-yl}-2,3-dihydroxy-cyclopentyl)-propionamide trifluoroacetate (Example 12) by replacing N-((1S,2R,3S,4R)-4-{6-[2,2-bis-(4-methoxy-phenyl)-ethylamino]-2-chloro-purin-9-yl}-2,3-dihydroxy-cyclopentyl)-propionamide with N-((1S,2R,3S,4R)-4-{2-chloro-6-[(2′-cyano-biphenyl-4-ylmethyl)-amino]-purin-9-yl}-2,3-dihydroxy-cyclopentyl)-propionamide trifluoroacetate (prepared analogously to Example 1 with the appropriate amine) and by replacing (3R)-3-(Boc-amino)pyrrolidine with the appropriate amine.

### Example 46-49

These compounds namely,
N-((1S,2R,3S,4R)-2,3-dihydroxy-4-{2-((S)-1-hydroxymethyl-2-phenyl-ethylamino)-6-[2-(4-sulfamoyl-phenyl)-ethylamino]-purin-9-yl}-cyclopentyl)-propionamide trifluoroacetate (Example 46),
(1R,2S,3R,5S)-3-[2-(4-amino-cyclohexylamino)-6-(2,2-diphenyl-ethylamino)-purin-9-yl]-5-(4-ethyl-[1,2,3]triazol-2-yl)-cyclopentane-1,2-diol trifluoroacetate (Example 47),
N-((1S,2R,3S,4R)-2,3-dihydroxy-4-{2-[2-(1H-imidazol-4-yl)ethylamino]-6-[2-(4-sulfamoyl-phenyl)-ethylamino]-purin-9-yl}-cyclopentyl)-propionamide trifluoroacetate (Example 48) and
N-((1S,2R,3S,4R)-4-{2-[((R)-1-ethyl-pyrrolidin-2-ylmethyl)-amino]-6-[2-(4-sulfamoyl-phenyl)-ethylamino]-purin-9-yl}-2,3-dihydroxy-cyclopentyl)-propionamide trifluoroacetate (Example 49),
   are prepared analogously to N-((1S,2R,3S,4R)-4-{2-((R)-3-amino-pyrrolidin-1-yl)-6-[2,2-bis-(4-methoxy-phenyl)-ethylamino]-purin-9-yl}-2,3-dihydroxy-cyclopentyl)-propionamide trifluoroacetate (Example 12) by replacing N-((1S,2R,3S,4R)-4-{6-[2,2-bis-4-methoxy-phenyl)ethylamino]-2-chloro-purin-9-yl}-2,3-dihydroxy-cyclopentyl)-propionamide with N-((1S,2R,3S,4R)-4-{2-chloro-6-[2-(4-sulfamoyl-phenyl)-ethylamino]-purin-9-yl}-2,3-dihydroxy-cyclopentyl)-propionamide trifluoroacetate (Example 3) and by replacing (3R)-3-(Boc-amino)pyrrolidine with the appropriate amine.

### Example 50

### N-((1S,2R,3S,4R)-4-{2-((R)-3-Amino-pyrrolidin-1-yl)-6-[2,2-bis-(4-hydroxy-phenyl)-ethylamino]-purin-9-yl}-2,3-dihydroxy-cyclopentyl)-propionamide trifluoroacetate

This compound is prepared analogously to N-((1S,2R,3S,4R)-4-{2-((R)-3-amino-pyrrolidin-1-yl)-6-[2,2-bis-(4-methoxy-phenyl)-ethylamino]-purin-9-yl}-2,3-dihydroxy-cyclopentyl)-propionamide trifluoroacetate (Example 12) by replacing N-((1S,2R,3S,4R)-4-{6-[2,2-bis-(4-methoxy-phenyl)-ethylamino]-2-chloro-purin-9-yl}-2,3-dihydroxy-cyclopentyl)-propionamide with N-((1S,2R,3S,4R)-4-{6-[2,2-Bis-(4-hydroxy-phenyl)-ethylamino]-2-chloro-purin-9-yl}-2,3-dihydroxy-cyclopentyl)-propionamide (Example 33).

### Example 51

### N-((1S,2R,3S,4R)-4-{6-[2,2-Bis-(4-methoxy-phenyl)-ethylamino]-2-[(R)-3-(3-pyridin-4-yl-ureido)-pyrrolidin-1-yl]-purin-9-yl}-2,3-dihydroxy-cyclopentyl)-propionamide

The title compound is prepared analogously to Example 35 by replacing N-((1S,2R,3S,4R)-4-{2-((R)-3-amino-pyrrolidin-1-yl)-6-[2,2-bis-(4-hydroxy-phenyl)-ethylamino]-purin-9-yl}-2,3-dihydroxy-cyclopentyl)-propionamide trifluoroacetate with N-((1S,2R,3S,4R)-4-{2-((R)-3-amino-pyrrolidin-1-yl)-6-[2,2-bis-(4-methoxy-phenyl)-ethylamino]-purin-9-yl}-2,3-dihydroxy-cyclopentyl)-propionamide.

### Example 52

### ((R)-1-{6-[2,2-Bis-4-fluoro-phenyl)-2-hydroxy-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)carbamic acid tert-butyl ester

### Step 1: Acetic acid (2R,3R,4R,5R)-4-acetoxy-2-{6-[2,2-bis(4-fluoro-phenyl)-2-hydroxy-ethylamino]-2-chloro-purin-9-yl}-5-(2-ethyl-2H-tetrazol-5-yl)-tetrahydro-furan-3-yl ester

A suspension of 2-amino-1,1-bis-(4-fluoro-phenyl)-ethanol (Intermediate T) (151 mg, 0.61 mmol), acetic acid (2R,3R,4R,5R)-4-acetoxy-2-(2,6-dichloro-purin-9-yl)-5-(2-ethyl-2H-tetrazol-5-yl)-tetrahydro-furan-3-yl ester (WO 9828319) (288 mg, 0.61 mmol) and DIPEA (106 μl, 0.61 mmol) in THF (2 ml) is stirred at 50 C for 8 hr. The reaction mixture is concentrated in vacuo and the residues partitioned between ethyl acetate and 0.1M HCl. The organic portion is separated, dried (Na₂SO₄) and concentrated in vacuo to give a foam. Purification by C-18 reverse phase column chromatography eluting with acetonitrile:water (0.1% TFA) to afford the titled compound (147 mg).

### Step 2: (2R,3R,4S,5R)-2-{6-[2,2-Bis-(4-fluoro-phenyl)-2-hydroxy-ethylamino]-2-chloro-purin-9-yl}-5-(2-ethyl-2H-tetrazol-5-yl)-tetrahydro-furan-3,4-diol

A mixture comprising acetic acid (2R,3R,4R,5R)-4-acetoxy-2-{6-[2,2-bis-(4-fluoro-phenyl)-2-hydroxy-ethylamino]-2-chloro-purin-9-yl}-5-(2-ethyl-2H-tetrazol-5-yl-tetrahydro-furan-3-yl ester (Step 1) (136 mg, 0.20 mmol) and potassium carbonate (1 ml of 10% aqueous solution) in methanol (5 ml) is stirred at RT overnight. The reaction mixture is concentrated in vacuo and the residues partitioned between DCM/water. The organic portion is separated, dried (Na₂SO₄) and concentrated in vacuo to afford the titled compound. (97 mg).

### Step 3: ((R)-1-{6-[2,2-Bis-(4-fluoro-phenyl)-2-hydroxy-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-carbamic acid tert-butyl ester

(2R,3R,4R,5R)-4-acetoxy-2-{6-[2,2-bis-(4-fluoro-phenyl)-2-hydroxy-ethylamino]-2-chloro-purin-9-yl}-5-(2-ethyl-2H-tetrazol-5-yl)tetrahydro-furan-3-yl ester (Step 2) (87 mg, 0.13 mmol), (R)-pyrrolidin-3-yl-carbamic acid tert-butyl ester (101 mg, 0.54 mmol) and sodium iodide (20 mg, 0.13 mmol) are dissolved in acetonitrile (0.5 ml) and NMP (0.5 ml) under an inert atmosphere of Argon. The reaction mixture is heated using microwave radiation in a Personal Chemistry Emrys™ Optimizer microwave reactor at 160° C. for 1 hour. The reaction mixture is diluted with EtOAc/water and the pH is adjusted to pH2 using 1M HCl. The organic portion is separated, washed with water twice, dried (MgSO₄) and concentrated in vacuo to afford the titled compound as a brown foam.

### Example 53

### (2R,3R,4S,5R)-2-{2-((R)-3-Amino-pyrrolidin-1-yl)-6-[2,2-bis-(4-fluoro-phenyl)-2-hydroxy-ethylamino]-purin-9-yl}-5-(2-ethyl-2H-tetrazol-5-yl)-tetrahydro-furan-3,4-diol

((R)-1-{6-[2,2-Bis-(4-fluoro-phenyl)-2-hydroxy-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-carbamic acid tert-butyl ester (Example 52) is dissolved in DCM and TFA and stirred at RT overnight. The reaction mixture is concentrated in vacuo and purified by C-18 reverse phase column chromatography eluting with acetonitrile:water (0.1% TFA) (gradient 0-100% acetonitrile) to afford the titled compound.

### Example 54

### ((R)-1-{6-[2,2-Bis-(4-fluoro-phenyl)-2-hydroxy-ethylamino]-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-carbamic acid tert-butyl ester

(2R,3R,4S,5R)-2-{2-((R)-3-Amino-pyrrolidin-1-yl)-6-[2,2-bis-(4-fluoro-phenyl)-2-hydroxy-ethylamino]-purin-9-yl}-5-(2-ethyl-2H-tetrazol-5-yl)-tetrahydro-furan-3,4-diol (Example 53) and pyridin-3-yl-carbamic acid phenyl ester (Intermediate ZB) are dissolved in acetonitrile. The reaction mixture is heated using microwave radiation in a Personal Chemistry Emrys™ Optimizer microwave reactor at 110° C. for 1 hr. The solvent is remove in vacuo and the product obtained from the resulting residues by trituration with EtOAc.

### Example 55

### 1-((R)-1-{6-[2,2-Bis-(4-hydroxy-phenyl)-ethylamino]-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-3-yl-urea

### Step 1: Acetic acid (2S,3R,4R,5R)-4-acetoxy-5-{6-[2,2-bis-(4-hydroxy-phenyl)-ethylamino]-2-chloro-purin-9-yl}-2-(3-ethyl-isoxazol-5-yl)-tetrahydro-furan-3-yl ester

A mixture comprising acetic acid (2S,3R,4R,5R)-4-acetoxy-5-(2,6-dichloro-purin-9-yl)-2-(3-ethyl-isoxazol-5-yl)-tetrahydro-furan-3-yl ester (WO 99/38877), 4,4′-(2-aminoethylidene)bis-phenol (Intermediate E) and DIPEA in DCE is stirred under an inert atmosphere of Argon at 50° C. overnight. After cooling to RT, 0.1 M HCl is added, the organic portion separated and concentrated in vacuo to afford the titled compound which is used in the next step without further purification.

### Step 2: (2R,3R,4S,5S)-2-{6-[2,2-Bis-(4-hydroxy-phenyl)-ethylamino]-2-chloro-purin-9-yl}-5-(3-ethyl-isoxazol-5-yl)-tetrahydro-furan-3,4-diol

A solution of acetic acid (2S,3R,4R,5R)-4-acetoxy-5-{6-[2,2-bis-(4-hydroxyphenyl)-ethylamino]-2-chloro-purin-9-yl}-2-(3-ethyl-isoxazol-5-yl)-tetrahydro-furan-3-yl ester (step 1) in MeOH/Chloroform (3:1 MeOH/Chloroform) is treated with saturated potassium carbonate solution. After stirring at RT overnight, the reaction is diluted with DCM/water and the organic portion is separated. The organic portion is concentrated in vacuo to afford the titled compound.

### Step 3: (2R,3R,4S,5S)-2-[6-[2,2-Bis-(4-hydroxy-phenyl)-ethylamino]-2-((R)-3-BOC-amino-pyrrolidin-1-yl)-purin-9-yl]-5-(3-ethyl-isoxazol-5-yl)-tetrahydro-furan-3,4-diol

(2R,3R,4S,5S)-2-{6-[2,2-Bis(4-hydroxy-phenyl)-ethylamino]-2-chloro-purin-9-yl}-5-(3-ethyl-isoxazol-5-yl)-tetrahydro-furan-3,4-diol (step 2) (1 g, 1.73 mmol), (3R-3-(BOC-amino)pyrrolidine (965 mg, 5.18 mmol) and sodium iodide (259 mg, 1.73 mmol) are dissolved in acetonitrile (10 ml) and NMP (0.5 ml). The reaction mixture is heated using microwave radiation at 160° C. for 30 minutes in the Personal Chemistry Emrys™ Optimizer microwave reactor. The reaction mixture is concentrated in vacuo and purified by C-18 reverse phase column chromatography eluting with acetonitrile:water (0.1% TFA) (gradient 0-100% acetonitrile) to afford the titled compound.

### Step 4: (2R,3R,4S,5R)-2-{2-((R)-3-Amino-pyrrolidin-1-yl)-6-[2,2-bis-(4-hydroxy-phenyl)-ethylamino]-purin-9-yl}-5-(2-ethyl-2H-tetrazol-5-yl)tetrahydro-furan-3,4-diol

(2R,3R,4S,5S)-2-[6-[2,2-Bis-4-hydroxy-phenyl)-ethylamino]-2-((R-3-BOC-amino-pyrrolidin-1-yl)-purin-9-yl]-5-(3-ethyl-isoxazol-5-yl)-tetrahydro-furan-3,4-diol (step 3) is dissolved in DCM and TFA and stirred at RT overnight. The reaction mixture is concentrated in vacuo to afford the titled compound.

### Step 5: 1-((R)-1-{6-[2,2-Bis-(4-hydroxy-phenyl)-ethylamino]-9-[(2R,3R,4S,5S)-5-(3-ethyl-isoxazol-5-yl)-3,4-dihydroxy-tetrahydro-furan-2-yl]-9H-purin-2-yl}-pyrrolidin-3-yl)-3-pyridin-3-yl-urea

(2R,3R,4S,5R)-2-{2-((R)-3-Amino-pyrrolidin-1-yl)-6-[2,2-bis-(4-hydroxy-phenyl)-ethylamino]-purin-9-yl}-5-(2-ethyl-2H-tetrazol-5-yl)-tetrahydro-furan-3,4-diol (step 4) and Pyridin-3-yl-carbamic acid phenyl ester (Intermediate ZB) are dissolved in methanol and TEA. The reaction mixture is heated using microwave radiation at 100° C. for 30 minutes in the Personal Chemistry Emrys™ Optimizer microwave reactor. The reaction mixture is concentrated in vacuo and purified by C-18 reverse phase column chromatography eluting with acetonitrile:water (0.1% TFA) (gradient 0-100% acetonitrile) to afford the titled compound.

### Example 56

### 1-((R)-1-{6-((S)-1-Benzyl-2-hydroxy-ethylamino)-9-[(2R,3R,4S,5R)-5-(2-ethyl-2H-tetrazol-5-yl)-3,4-dihydroxy-tetrahydro-furan-2-yl]-9H-purin-2-yl}-pyrrolidin-3-yl)-3-pyridin-3-yl-urea

This compound is prepared analogously to Example 55 by replacing acetic acid (2S,3R,4R,5R)-4-acetoxy-5-(2,6-dichloro-purin-9-yl)-2-(3-ethyl-isoxazol-5-yl)-tetrahydro-furan-3-yl ester (WO 99/38877) with acetic acid (2R,3R,4R,5R)-4-acetoxy-5-(2,6-dichloro-purin-9-yl)-2-(2-ethyl-2H-tetrazol-5-yl)tetrahydro-furan-3-yl ester (WO 98/28319) and by replacing 2,2-diphenylethylamine with L-phenylalaminol.

1. A compound of formula (I) or stereoisomers or pharmaceutically acceptable salts thereof, 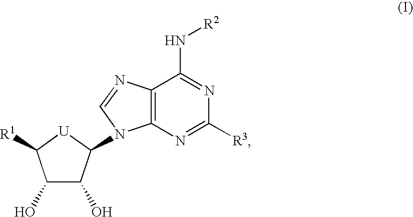
wherein
U is selected from CH₂ and O, with the proviso that when U is O then R¹ is not a N-bonded substituent;
R¹ is a 3- to 12-membered heterocyclic group containing from 1 to 4 ring nitrogen atoms and optionally containing from 1 to 4 other heteroatoms selected from the group consisting of oxygen and sulfur, that group being optionally substituted by oxo, C₁-C₈-alkoxy, C₆-C₁₀-aryl, R^{1a} or by C₁-C₈-alkyl optionally substituted by hydroxyl, or
R¹ is —NH₂, —NH—C₁-C₈-alkylcarbonyl, —NH—C₃-C₈-cycloalkylcarbonyl, —NHSO₂—C₁-C₈-alkyl, —NH—C₇-C₁₄-aralkylcarbonyl or —NHC(═O)—C(═O)—NH—C₁-C₈-alkyl optionally substituted by R^{1a}, or
R¹ is selected from CH₂OH, CH₂—O—C₁-C₈-alkyl, C(O)—O—C₁-C₈-alkyl, C(O)NH₂, and C(O)—NH—C₁-C₈-alkyl;
R^{1a} is a 3- to 12-membered heterocyclic ring containing at least one ring heteroatom selected from the group consisting of nitrogen, oxygen and sulfur, said 3- to 12-membered heterocyclic ring being optionally substituted by halo, cyano, oxo, hydroxy, carboxy, amino, nitro, C₁-C₈-alkyl, C₁-C₈-alkylsulfonyl, aminocarbonyl, C₁-C₈-alkylcarbonyl or C₁-C₈-alkoxy optionally substituted by aminocarbonyl;
R² is C₁-C₈-alkyl substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, a C₃-C₁₅-carbocyclic group optionally substituted by O—C₇-C₁₄ aralkyl, C₃-C₁₅-carbocyclic group, O—C₁-C₈-alkyl, C₂-C₈-alkenyl, C₂-C₈-alkynyl or C₁-C₈-alkyl, O—C₁-C₈-alkyl, —SO₂—C₁-C₈-alkyl, a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl, with the proviso that R² is not 2,2,diphenyl-ethyl, or
R² is a C₃-C₁₅-carbocyclic group optionally substituted by O—C₇-C₁₄ aralkyl, C₃-C₁₅-carbocyclic group, O—C₁-C₈-alkyl, or C₁-C₈-alkyl, or
R² is a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl;
R³ is hydrogen, halo, C₂-C₈-alkenyl, C₂-C₈-alkynyl, C₁-C₈-alkoxycarbonyl, C₁-C₈-alkyl optionally substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, or
R³ is amino optionally substituted by C₃-C₈-cycloalkyl optionally substituted by amino, hydroxy, C₇-C₁₄-aralkyloxy, —SO₂—C₆-C₁₀-aryl or —NH—C(═O)—NH—R^{3c}, or
R³ is amino substituted by R^{3a}, —R^{3a}—C₇-C₁₄-aralkyl or a C₅-C₁₅-carbocyclic group optionally substituted by OH, C₁-C₈-alkyl or C₁-C₈-alkoxycarbonyl, or
R³ is aminocarbonyl optionally substituted by R^{3b}, or
R³ is C₁-C₈-alkylamino optionally substituted by OH, R^{3b}, amino, di(C₁-C₈-alkyl)amino, —NH—C(═O)—C₁-C₈-alkyl, —NH—SO₂—C₁-C₈-alkyl, —NH—C(═O)—NH—R^{3c}, —NH—C(═O)—NH—C₁-C₈-alkyl-R^{3b}, a C₅-C₁₅-carbocyclic group or by C₆-C₁₀-aryl optionally substituted by C₆-C₁₀-aryloxy, or
R³ is C₁-C₈-alkylaminocarbonyl or C₃-C₈-cycloalkylamino-carbonyl optionally substituted by amino, C₁-C₈-alkylamino, di(C₁-C₈-alkyl)amino or —NH—C(═O)—NH—R^{3d}, or
R³ is a 3- to 12-membered heterocyclic group containing from 1 to 4 ring nitrogen atoms and optionally containing from 1 to 4 other heteroatoms selected from the group consisting of oxygen and sulfur, that group being optionally substituted by 0-3R⁴;
R^{3a} and R^{3b} are each independently a 3- to 12-membered heterocyclic group containing at least one ring heteroatom selected from the group consisting of nitrogen, oxygen and sulfur; optionally substituted by halo, cyano, oxo, OH, carboxy, nitro, C₁-C₈-alkyl, C₁-C₈-alkylcarbonyl, OH—C₁-C₈-alkyl, C₁-C₈-haloalkyl, amino-C₁-C₈-alkyl, amino(OH)C₁-C₈-alkyl and C₁-C₈-alkoxy optionally substituted by aminocarbonyl;
R^{3c} is a 5- or 6-membered heterocyclic group containing at least one ring heteroatom selected from the group consisting of nitrogen, oxygen and sulfur, which is optionally substituted by a 5- or 6-membered heterocyclic group containing at least one ring heteroatom selected from the group consisting of nitrogen, oxygen and sulfur;
R^{3d} are independently a 5- or 6-membered heterocyclic ring containing at least one ring heteroatom selected from the group consisting of nitrogen, oxygen and sulfur, said 5- or 6-membered heterocyclic ring being optionally substituted by halo, cyano, oxo, OH, carboxy, amino, nitro, C₁-C₈-alkyl, C₁-C₈-alkylsulfonyl, aminocarbonyl, C₁-C₈-alkylcarbonyl, C₁-C₈-alkoxy optionally substituted by aminocarbonyl, or a 5- or 6-membered heterocyclic ring containing at least one ring heteroatom selected from the group consisting of nitrogen, oxygen and sulfur, said ring also being optionally substituted by halo, cyano, oxo, OH, carboxy, amino, nitro, C₁-C₈-alkyl, C₁-C₈-alkylsulfonyl, aminocarbonyl, C₁-C₈-alkylcarbonyl, C₁-C₈-alkoxy optionally substituted by aminocarbonyl;
R⁴ is selected from OH, C₁-C₈-alkyl optionally substituted by OH, C₁-C₈-alkoxy, C₇-C₁₄-aralkyl optionally substituted with OH, O—C₁-C₈-alkyl, halogen C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, O—C₁-C₈-alkyl or -halogen, NR^{4a}R^{4b}, NHC(O)R^{4c}, NHS(O)₂R^{4d}, NHS(O)₂R^{4c}, NR^{4f}C(O)NR^{4e}R^{4h}, NR⁴ⁱC(O)OR^{4j}, C₁-C₈-alkylcarbonyl, C₁-C₈-alkoxycarbonyl, di(C₁-C₈-alkyl)aminocarbonyl, COOR^{4k}, C(O)R^{4l}, and a 3- to 12-membered heterocyclic group containing at least one ring heteroatom selected from the group consisting of nitrogen, oxygen and sulfur optionally substituted by COOR^{4p};
R^{4a}, R^{4b}, R^{4c}, R^{4f}, R^{4h} and R⁴ⁱ are, independently, H, or C₁-C₈-alkyl;
R^{4d}, R^{4e}, and R^{4j} are, independently, C₁-C₈-alkyl or a 3- to 12-membered heterocyclic group containing at least one ring heteroatom selected from the group consisting of nitrogen, oxygen and sulfur, optionally substituted by 0-3R⁵;
R^{4k} is H, C₁-C₈-alkyl, C₆-C₁₀-aryl or a 3- to 12-membered heterocyclic group containing at least one ring heteroatom selected from the group consisting of nitrogen, oxygen and sulfur;
R^{4l} is C₁-C₈-alkyl, C₆-C₁₀-aryl, NHR⁶ or a 3- to 12-membered heterocyclic group containing at least one ring heteroatom selected from the group consisting of nitrogen, oxygen and sulfur;
R⁵ is selected from OH, C₁-C₈-alkyl optionally substituted by OH, SO₂R¹⁰, C₇-C₁₄-aralkyl optionally substituted with OH, O—C₁-C₈-alkyl, C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, NR^{5a}R^{5b}, NHC(O)R^{5c}, NHS(O)₂R^{5d}, NHS(O)₂R^{5e}, NR^{5f}C(O)NR^{5g}R^{5h}, NR⁵ⁱC(O)OR^{5j}, C₁-C₈-alkylcarbonyl, C₁-C₈-alkoxycarbonyl, di(C₁-C₈-alkyl)aminocarbonyl, COOR^{5k}, C(O)R^{5l}, C(O)NHR^{5m} or a 3- to 12-membered heterocyclic group containing at least one ring heteroatom selected from the group consisting of nitrogen, oxygen and sulfur, optionally substituted by 0-3R⁷;
R^{5a}, R^{5b}, R^{5c}, R^{5f}, R^{5h} and R⁵ⁱ are, independently, H, C₁-C₈-alkyl or C₆-C₁₀-aryl;
R^{5d}, R^{5e}, R^{5g}, R^{5j} and R^{5m} are, independently, C₁-C₈-alkyl or a 3- to 12-membered heterocyclic group containing at least one ring heteroatom selected from the group consisting of nitrogen, oxygen and sulfur, optionally substituted by COOR⁸;
R^{5k} is H, C₁-C₈-alkyl, C₆-C₁₀-aryl or a 3- to 12-membered heterocyclic group containing at least one ring heteroatom selected from the group consisting of nitrogen, oxygen and sulfur;
R^{5l} is C₁-C₈-alkyl, C₆-C₁₀-aryl or a 3- to 12-membered heterocyclic group containing at least one ring heteroatom selected from the group consisting of nitrogen, oxygen and sulfur, optionally substituted by COOR⁹;
R⁶ is COOR^{6a} or a 3- to 12-membered heterocyclic group containing at least one ring heteroatom selected from the group consisting of nitrogen, oxygen and sulfur, optionally substituted by COOR^{6b};
R^{6a}, R^{6b}, R⁷, R⁸ and R⁹ are selected from H, C₁-C₈-alkyl and C₇-C₁₄-aralkyl; and
R¹⁰ is C₁-C₈-alkyl optionally substituted by halogen, C₆-C₁₀-aryl optionally substituted by OH, C₁-C₈-alkyl, O—C₁-C₈-alkyl or -halogen.

2. A compound according to claim 1 of formula (I) or stereoisomers or pharmaceutically acceptable salts thereof,
wherein
U is selected from CH₂ and O, with the proviso that when U is O then R¹ is not a N-bonded substituent;
R¹ is a 3- to 12-membered heterocyclic group containing from 1 to 4 ring nitrogen atoms and optionally containing from 1 to 4 other heteroatoms selected from the group consisting of oxygen and sulfur, that group being optionally substituted by C₁-C₈-alkyl optionally substituted by hydroxyl, or
R¹ is —NH₂, —NH—C₁-C₈-alkylcarbonyl, —NH—C₃-C₈-cycloalkylcarbonyl, —NHSO₂—C₁-C₈-alkyl, —NH—C₇-C₁₄-aralkylcarbonyl or —NHC(═O)—C(═O)—NH—C₁-C₈-alkyl optionally substituted by R^{1a};
R^{1a} is a 5- or 6-membered heterocyclic ring containing at least one ring heteroatom selected from the group consisting of nitrogen, oxygen and sulphur, said 5- or 6-membered heterocyclic ring being optionally substituted by halo, cyano, oxo, OH, carboxy, amino, nitro, C₁-C₈-alkyl, C₁-C₈-alkylsulfonyl, aminocarbonyl, C₁-C₈-alkylcarbonyl or C₁-C₈-alkoxy optionally substituted by aminocarbonyl;
R² is C₁-C₈-alkyl substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, a C₃-C₁₅-carbocyclic group optionally substituted by O—C₇-C₁₄ aralkyl, C₃-C₁₅-carbocyclic group, O—C₁-C₈-alkyl, C₂-C₈-alkenyl, C₂-C₈-alkynyl or C₁-C₈-alkyl, O—C₁-C₈-alkyl, —SO₂—C₁-C₈-alkyl, a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl, or
R² is a C₃-C₁₅-carbocyclic group optionally substituted by O—C₇-C₁₄ aralkyl, C₃-C₁₅-carbocyclic group, O—C₁-C₈-alkyl, or C₁-C₈-alkyl, or
R² is a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl;
R³ is hydrogen, halo, C₂-C₈-alkenyl, C₂-C₈-alkynyl, C₁-C₈-alkoxycarbonyl, C₁-C₈-alkyl optionally substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, or
R³ is amino optionally substituted by C₃-C₁₅-carbocyclic group optionally substituted by amino, or
R³ is C₁-C₈-alkylamino optionally substituted by OH, R^{3b}, amino, di(C₁-C₈-alkyl)amino, —NH—C(═O)—C₁-C₈-alkyl, —NH—SO₂—C₁-C₈-alkyl, —NH—C(═O)—NH—R^{3c}, —NH—C(═O)—NH—C₁-C₈-alkyl-R^{3b}, a C₅-C₁₅-carbocyclic group or by C₆-C₁₀-aryl optionally substituted by C₆-C₁₀-aryloxy, or
R³ is a 3- to 12-membered heterocyclic group containing from 1 to 4 ring nitrogen atoms and optionally containing from 1 to 4 other heteroatoms selected from the group consisting of oxygen and sulfur, that group being optionally substituted by 0-3R⁴;
R^{3b} are each independently a 3- to 12-membered heterocyclic group containing at least one ring heteroatom selected from the group consisting of nitrogen, oxygen and sulfur; optionally substituted by halo, cyano, oxo, OH, carboxy, nitro, C₁-C₈-alkyl, C₁-C₈-alkylcarbonyl, OH—C₁-C₈-alkyl, C₁-C₈-haloalkyl, amino-C₁-C₈-alkyl, amino(OH)C₁-C₈-alkyl and C₁-C₈-alkoxy optionally substituted by aminocarbonyl;
R^{3c} is a 5- or 6-membered heterocyclic group containing at least one ring heteroatom selected from the group consisting of nitrogen, oxygen and sulfur, which is optionally substituted by a 5- or 6-membered heterocyclic group containing at least one ring heteroatom selected from the group consisting of nitrogen, oxygen and sulfur;
R⁴ is selected from OH, C₁-C₈-alkyl optionally substituted by OH, C₁-C₈-alkoxy, C₇-C₁₄-aralkyl optionally substituted with OH, O—C₁-C₈-alkyl, halogen C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, O—C₁-C₈-alkyl or -halogen, NR^{4a}R^{4b}, NHC(O)R^{4c}, NR^{4f}C(O)NR^{4e}R^{4h};
R^{4a}, R^{4b}, R^{4c}, R^{4f}, and R^{4h} are, independently, H, or C₁-C₈-alkyl;
R^{4e} is C₁-C₈-alkyl or a 3- to 12-membered heterocyclic group containing at least one ring heteroatom selected from the group consisting of nitrogen, oxygen and sulfur, optionally substituted by 0-3R⁵;
R⁵ is selected from OH, C₁-C₈-alkyl optionally substituted by OH, SO₂R¹⁰, C₇-C₁₄-aralkyl optionally substituted with OH, O—C₁-C₈-alkyl, C₆-C₁₀-aryl, or O—C₆-C₁₀-aryl, C₁-C₈-alkoxy, C₆-C₁₀-aryl optionally substituted by OH, C₁-C₈-alkyl, O—C₁-C₈-alkyl or -halogen, O—C₆-C₁₀-aryl optionally substituted by OH, C₁-C₈-alkyl, NR^{5a}R^{5b}, NHC(O)R^{5c}, NHS(O)₂R^{5d}, NHS(O)₂R^{5e}, NR^{5f}C(O)NR^{5g}R^{5h}, NR⁵ⁱC(O)OR^{5j}, C₁-C₈-alkylcarbonyl, C₁-C₈-alkoxycarbonyl, di(C₁-C₈-alkyl)aminocarbonyl, COOR^{5k}, C(O)R^{5l}, C(O)NHR^{5m}, or a 3- to 12-membered heterocyclic group containing at least one ring heteroatom selected from the group consisting of nitrogen, oxygen and sulfur, optionally substituted by 0-3R⁷;
R^{5a}, R^{5b}, R^{5c}, R^{5f}, R^{5h}, and R⁵ⁱ are, independently, H, C₁-C₈-alkyl or C₆-C₁₀-aryl;
R^{5d}, R^{5e}, R^{5g}, R^{5j} and R^{5m} are, independently, C₁-C₈-alkyl or a 3- to 12-membered heterocyclic group containing at least one ring heteroatom selected from the group consisting of nitrogen, oxygen and sulfur, optionally substituted by COOR⁸;
R^{5k} is H, C₁-C₈-alkyl, C₆-C₁₀-aryl or a 3- to 12-membered heterocyclic group containing at least one ring heteroatom selected from the group consisting of nitrogen, oxygen and sulfur;
R^{5l} is C₁-C₈-alkyl, C₆-C₁₀-aryl or a 3- to 12-membered heterocyclic group containing at least one ring heteroatom selected from the group consisting of nitrogen, oxygen and sulfur, optionally substituted by COOR⁹;
R⁶ is COOR^{6a} or a 3- to 12-membered heterocyclic group containing at least one ring heteroatom selected from the group consisting of nitrogen, oxygen and sulfur, optionally substituted by COOR^{6b};
R^{6a}, R^{6b}, R⁷, R⁸ and R⁹ are selected from H, C₁-C₈-alkyl and C₇-C₁₄-aralkyl; and
R¹⁰ is C₁-C₈-alkyl optionally substituted by halogen, C₆-C₁₀-aryl optionally substituted by OH, C₁-C₈-alkyl, O—C₁-C₈-alkyl or -halogen.

3. A compound according to claim 1 of formula (I) or stereoisomers or pharmaceutically acceptable salts thereof,
U is selected from CH₂ and O, with the proviso that when U is O then R¹ is not a N-bonded substituent;
R¹ is a 3- to 12-membered heterocyclic group containing from 1 to 4 ring nitrogen atoms and optionally containing from 1 to 4 other heteroatoms selected from the group consisting of oxygen and sulfur, that group being optionally substituted by C₁-C₈-alkyl, or
R¹ is —NH—C₁-C₈-alkylcarbonyl, —NH—C₃-C₈-cycloalkylcarbonyl, —NHSO₂—C₁-C₈-alkyl, —NH—C₇-C₁₄-aralkylcarbonyl or —NHC(═O)—C(═O)—NH—C₁-C₈-alkyl;
R² is C₁-C₈-alkyl substituted by OH, a C₃-C₁₅-carbocyclic group, C₆-C₁₀ aryl optionally substituted by OH, O—C₁-C₈-alkyl, CN, halogen, SO₂NH₂, SCH₃, a C₆-C₁₀-aryl, or O—C₇-C₁₄-aralkyl, or
R² is C₁-C₈-alkyl substituted by C₃-C₁₅-Carbocyclic group optionally substituted by C₂-C₈-alkenyl, or
R² is a C₃-C₁₅-carbocyclic group optionally substituted by a C₃-C₁₅-carbocyclic group, a C₇-C₁₄ aralkyl, or O—C₇-C₁₄-aralkyl, or
R² is a 3- to 12-membered heterocyclic group containing from 1 to 4 ring nitrogen atoms and optionally containing from 1 to 4 other heteroatoms selected from the group consisting of oxygen and sulfur, that group being optionally substituted by 3- to 12-membered heterocyclic group containing from 1 to 4 ring nitrogen atoms and optionally containing from 1 to 4 other heteroatoms selected from the group consisting of oxygen and sulfur, C₇-C₁₄ aralkyl, or C₆-C₁₄-aryl optionally substituted by O—C₇-C₁₄ aralkyl; and
R³ is hydrogen, halo, C₂-C₈-alkenyl, C₂-C₈-alkynyl, C₁-C₈-alkoxycarbonyl, C₁-C₈-alkyl optionally substituted by OH, halogen C₆-C₁₀-aryl optionally substituted by OH, SO₂R¹⁰, SC₁-C₈-alkyl, CN, halogen, O—C₇-C₁₄-aralkyl, or O—C₁-C₈-alkyl, a 3- to 12-membered heterocyclic group containing from 1 to 4 ring nitrogen atoms and optionally containing from 1 to 4 other heteroatoms selected from the group consisting of oxygen and sulfur, that group being optionally substituted by 0-3R⁴, C₁-C₈-alkylamino substituted by 3- to 12-membered heterocyclic group optionally substituted by a C₁-C₈-alkyl group, or an amino substituted by a C₃-C₁₅-carbocyclic group optionally substituted by an amine.

4. A compound according to claim 1 for use as a pharmaceutical.

5. A compound according to claim 1 in combination with an anti-inflammatory, bronchodilatory, antihistamine or anti-tussive drug substance, said compound and said drug substance being in the same or different pharmaceutical composition.

6. A composition comprising as active ingredient a compound according to claim 1, optionally together with a pharmaceutically acceptable diluent or carrier.

7. A composition according to claim 6, further comprising an anti-inflammatory, bronchodilatory, antihistamine or anti-tussive drug substance.

8. Use of a compound according to claim 1 for the manufacture of a medicament for the treatment of a condition mediated by activation of the adenosine A2a receptor.

9. Use of a compound according to claim 1 for the manufacture of a medicament for the treatment of an inflammatory or obstructive airways disease.

10. A method of preparing a compound of formula I as defined in claim 1 in free or salt form which comprises:
(i) reacting a compound of formula (II) 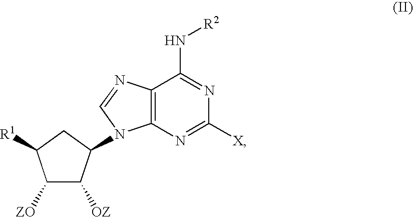
wherein
R¹, and R² are as defined in claim 1;
Z is H or a protecting group; and
X is a leaving group,
with a compound of formula (III)
H—R³ (III),
wherein
R³ is as defined in claim 1; and
(ii) removing any protecting groups and recovering the resultant compound of formula (I), in free or pharmaceutically acceptable salt form.
